(19) United States
(12) Patent Application Publication
(10) Pub. No.: US 2020/0138704 A1
(43) Pub. Date: May 7, 2020

(54) GELATIN GUMMY COMPOSTION AND METHODS OF MAKING AND USING THEREOF

(71) Applicant: Seattle Gummy Company, Seattle, WA (US)

(72) Inventors: Feng Wan, Issaquah, WA (US); William Brenden Carlson, Shoreline, WA (US)

(21) Appl. No.: 16/623,771

(22) PCT Filed: Jun. 20, 2018
(86) PCT No.: PCT/US2018/038484

§ 371 Date: Dec. 18, 2019

Related U.S. Application Data
(60) Provisional application No. 62/522,552, filed on Jun. 20, 2017.
(60) Provisional application No. 62/522,586, filed on Jun. 20, 2017.

(51) Int. Cl. A61K 9/00; A61K 31/485; A61K 47/42; A61K 47/36; A61K 47/26; A61K 31/138; A61K 31/4985; A61K 31/53; A61K 31/519; A61K 31/155; A61K 31/495; A61K 31/05; A61K 31/616; A61K 31/167; A61K 31/192; A61K 31/4545; A23G 3/36; A23G 3/42

(52) U.S. Cl. CPC A61K 9/0056; A23V 2002/00; A61K 47/42; A61K 47/36; A61K 47/26; A61K 31/138; A61K 31/4985; A61K 31/53; A61K 31/519; A61K 31/155; A61K 31/495; A61K 31/05; A61K 31/616; A61K 31/167; A61K 31/192; A61K 31/4545; A23G 3/368; A23G 3/42; A23G 3/364; A61K 31/485

(57) ABSTRACT

A method of making a gummy composition, comprising providing a gelatin blend, comprising gelatin and a swelling agent, providing a bonding blend, comprising a combination of at least two of a monosaccharide, a disaccharide and a tri-saccharide, providing an active blend, comprising an active agent and a liquid solvent, wherein the liquid solvent comprises a diprotic solvent, tri-protic solvent, or a combination thereof, combining the active blend, the bonding blend and a gelatin blend to provide a molding blend, and forming the molding blend into a gummy composition

### CROSS-REFERENCES TO RELATED APPLICATIONS

This application claims the benefit of priority from, and hereby incorporates by reference the entire disclosure, co-pending U.S. Provisional Application for Patent Ser. No. 62/522,552, filed Jun. 20, 2017 and U.S. Provisional Application for Patent Ser. No. 62/522,586, filed Jun. 20, 2017.

### TECHNICAL FIELD

The application relate generally to gummy composition or formula, methods of making, using, or administration gummy compositions, and kits comprising gummy compositions.

### BACKGROUND

Pharmaceuticals are available in a variety of composition dosages for treating the diseases. Dosages that are formulated to take orally including tablets, capsules, soft-gels, powders, chewable tablets, and liquid suspensions.

Tablets, capsules and soft-gels are difficult for individuals who have difficulties swallowing pills. This problem is magnified when the medications need to be taken 2-4 times per day to provide the desired therapeutic effect. Moreover, the need for a source of water or other liquid to assist with swallowing solid composition dosages can complicate administration.

Powders are often difficult to administrator and chewable tablets can be hard to chew especially for seniors and young children. In addition, powders and chewable tablets often have an unpleasant after-taste.

Liquid suspensions or solutions are sometimes used as an alternative to solid oral composition dosages. However, the dosing with liquid composition dosages is not precise, which can lead to the administration of too little or too much medications. In addition, liquid composition dosages are messy and often have a bitter taste, which could impact person compliance.

Absorption of drugs from the oral cavity into the mucosal tissues is typically a fast event. Dissolved drugs partition into the mucosal membranes and within minutes will reach equilibrium with drug in solution in the oral cavity.

These issues relating to the traditional composition dosage can lead to sub-optimal person compliance with taking medications resulting in the ineffective treatment and prolonged illness. The remains a need for alternative composition dosages for medications to treat various symptoms and illness.

### SUMMARY

In one aspect, the application provides methods of producing a gummy composition dosage. In one embodiment, the method includes the steps of providing a gelatin blend; providing a bonding blend; providing an active blend; combining the active blend, the bonding blend and a gelatin blend to provide a molding blend; and forming the molding blend into a gummy composition dosage.

Gelatin Blend

The gelatin blend may include gelatin and a swelling agent. The gelatin blend may include gelatin having a molecule weight (MW) from about 40 kDa to about 150 kDa. In one embodiment, the MW may be from 50 kDa to 100 kDa. In one embodiment, the MW may be more than 150 kDa. In one embodiment, the MW may be less than 40 Dka.

The gelatin blend may include from about 25% to about 45% by weight of gelatin. In one embodiment, the weight percentage of the gelatin is about 25%, 30%, 35%, 40% or 45% of the gelatin blend.

The swelling solvent may be any protic solvent. In one embodiment, the swelling solvent comprises water, an alcohol, or a combination thereof. In one embodiment, the alcohol may be ethanol, glycerol, or a combination thereof. In one embodiment, the swelling solvent comprises ethanol. In one embodiment, ethanol represents about from about 5% to about 30% weight of the gelatin blend. In one embodiment, the swelling solvent comprises glycerol.

The gelatin blend may further include a plasticizing agent. In one embodiment, the plasticizing agent serves to improve the texture or taste profile of the final products.

In one embodiment, the plasticizing agent may be a saccharide, a polysaccharide having a MW of not exceeding 3.6 kDa, a polyol, a fatty acid or salt, a lipid or fat, an oil, a gum, or a combination thereof. The saccharide may include less than 20 sugar monomers. For example, the saccharide may be a monosaccharide, a disaccharide, a trisacchride. The monosaccharide comprises glucose, fructose, galactose, lactose, mannose, allose, altrose, fructose, glucose, sorbose, tagatose, arabinose, lyxose, rhamnose, ribose, ribulose, sucroribulose, xylose, erythrose, erythrulose, threose, their isomers, or a combination thereof.

The polysaccharide may have a MW of not exceeding 20K Dalton. In one embodiment, the polysaccharide has a MW from about 900 to about 4,000 Dalton. The polysaccharide may include from about 3 to about 30 sugar monomers. In one embodiment, the polysaccharide comprise from about 6 to about 20 sugar monomers. In one embodiment, the polysaccharide comprises a maltodextrin, cyclodextrin, polydextrose, glucan, mannan, galactan or a combination thereof.

The polyol may include without limitation xylitol, erythiol, isomalt, lactitol, maltitol, mannitol, sorbitol, or a combination thereof.

The fatty acid may be a saturated fatty acid, a unsaturated fatty acid, or a combination thereof. In one embodiment, the fatty acid comprises a carbon chain having from about 2 to about 22 carbons. In one embodiment, the fatty acid and fatty acid triglyceride comprises palmitic acid, stearic acid, oleic acid, linoleic acid, linolenic acid, omega-3 fatty acid, DHA, EPA, or a combination thereof. In one embodiment, the oil comprises coconut oil, palm oil, palm kernel oil, corn oil, rapeseed oil, grape seed oil, cannabis oil, sunflower oil, avocado oil, clary sage oil, olive oil, chia seed oil, flaxseed oil, linseed oil, fish oil, algae oil, krill oil or a combination hereof.

The gum may be acadia gum, xanthum gum, lotus seed gum, gum Arabic or a combination thereof.

In one embodiment, the gelatin blend may be heated to from about 120° F. to about 180° F. before the combining step. In one embodiment, the gelatin blend is heated to about 140° F. before the combining step.

Bonding Blend

The bonding blend facilitates the formation of the gummy matrix. The bonding blend may include a combination of at least two of a monosaccharide, a disaccharide and a tri-saccharide. In one embodiment, the bonding blend comprises a disaccharide and a tri-saccharide. In one embodiment, the bonding blend comprises a monosaccharide and a tri-saccharide. In one embodiment, the boding blend comprises a monosaccharide, a disaccharide and a tri-saccharide. In one embodiment, the first and the second saccharide are in such a ration as to preventing the crystallization and co-crystallization of any of the saccharides.

In one embodiment, the monosaccharide may include glucose, mannose, fructose, sorbose, tagatose, psicose, ribose, galactose, xylose, lyxose, araabinose, ribulose, xylulose or a combination thereof. In one embodiment, the disaccharide may include lactose, trehalose, isomaltulose, isomaltulose, maltose, cellobiose, chitobiose, sucrose, isomaltose, nigerose, kojibiose, sophorose, gentiobiose, mannobiose, rutinulose, turanose, melibiose, melibiulose, lactulose, or a combination thereof. In one embodiment, the tri-saccharide may include raffinose, maltotriose, melezitose, isomaltotriose, kestose, nigertriose or a combination thereof.

In one embodiment, the bonding blend includes a monosaccharide and a disaccharide. In one embodiment, the monosaccharide and the disaccharide have a molar ratio from about 1:3 to about 3:1, and ratio in between. In one embodiment, the monosaccharide and the disaccharide have a molar ratio from about 1:1.5 to about 1.5:1, from about 1:2 to about 2:1, from about 1:1 to about 2:1, from about 1:3 to about 1:1, including any ratio in between.

In one embodiment, the bonding blend comprises a glucose syrup and sucrose. The glucose syrup may be corn syrup, tapioca syrup, rice syrup, barley syrup, cassava syrup, potato syrup or a combination thereof.

In one embodiment, the bonding blend may include tagatose and trehalose. In one embodiment, the bonding blend may include psicose and trehalose. In one embodiment, the bonding blend may include psicose and isomaltulose. In one embodiment, the bonding blend may include tagatose and isomaltulose. In one embodiment, the bonding blend may include psicose, sorbose, trehalose, isomaltulose, or any combination thereof. The surprising discovery through the extensive testing and research by the applicant shows that any combination of tagatose, sorbose, psicose, trehalost and palatinose (including a subset of at least two of these sugars) achieves a synergistically enhanced sweetness profile of the blend.

The bonding blend may represent from about 30% to about 80% of the gummy composition. In one embodiment, the bonding blend represents from about 50% to about 75% of the gummy composition. In one embodiment, the gummy composition represents from about 70% to about 75% of the gummy composition.

Because of the high percentage of the bonding blend in the gummy composition, the bonding blend may impart certain glycemic index(GI) value to the gummy composition. In one embodiment, the bonding blend comprises low GI saccharides resulting in a gummy composition having a GI value not exceeding 10, 12, 15, 16, 18, 20, 25, or 30. The bonding blend may impart a glycemic index (GI) value to the gummy composition. In one embodiment, the GI value of the gummy composition is less than 10, 12, 15, 18, 19, 20, 25, 30, 40, 50, 60, 70, 80 or 100.

In one embodiment, the bonding blend has a Brix number from about 85 to about 95 before the combining step. In one embodiment, the bonding blend has a Brix number from about 87 to about 92. In one embodiment, the bonding blend has a Brix number of about 90. In one embodiment, the bonding blend may a higher Brix number than the molding blend before the combining step.

In one embodiment, the bonding blend may be heated to from about 120° F. to about 180° F. before the combining step. In one embodiment, the bonding blend may be heated to from about 145° F. to about 150° F. In one embodiment, the bonding blend has a temperature from about 140° F. to about 150° F.

Active Blend and Active Agent

The active blend may include an active agent and a liquid solvent. In one embodiment, the active blend has a pH from about 5.5 to about 8.5 before the combining step. In one embodiment, the active blend has a pH from about 7.5 to about 8.

In one embodiment, the active blend may further include a neutralizing agent. In one embodiment, the neutralizing agent and the active agent are present at approximately equivalent ratio stoichiometrically. In one embodiment, the neutralizing agent comprises a basic agent. In one embodiment, the basic agent comprises an inorganic salt, an organic base, an amino acid, or a combination thereof. The inorganic salt may include NaOH, Na2CO3, NaHCO₃, KOH, KHCO3, K2CO3, Ca(HCO3)2, CaCO3, Ca(OH)2, MgCO3, Mg(OH)2, C6H5Na3O7, C6H5K3O7, Mg2(C6H5O7)2, Ca2(C6H5O7)2 or a combination thereof. In one embodiment, the organic base may be an organic compound having a primary or second amine group. In one embodiment, the organic base may be aspartame, glucosamine, caffeine, theobromine, xanthine, xanthine derivatives, nucleic acid base, or a combination thereof. In one embodiment, the amino acid comprises lysine, tryptophan, leucine, isoleucine, glutamine, glycine, methionine, cysteine, taurine, tyrosine, arginine, aspartic acid, tryptophan or a combination thereof.

The liquid solvent may include a diprotic solvent, tri-protic solvent, or a combination thereof. The diprotic solvent may include water, propylene glycol, 1, 8-octanediol, 1,2-octanediol, 1,2-decanediol, 1,10-decanediol, or a combination thereof. The tri-protic solvent may include phosphoric acid, 1,2,3-propantriol, glycerol, glycerin, or a combination thereof.

The combining step comprises mixing the gelatin blend and the bonding blend to provide a matrix blend having a Brix number from about 85 to about 95 and combining the matrix blend with the active blend to provide the molding blend. In one embodiment, the combining step comprises mixing the bonding blend and the active blend to provide a bonding active blend having a Brix number from about 85 to about 95 and combining the bonding active blend with the gelatin blend to provide the molding blend. In one embodiment, the combining step comprises mixing the gelatin blend with the active blend to provide a gelatin active blend having a Bix number from about 85 to about 95 and combining the gelatin active blend with the bonding blend to provide the molding blend.

Molding Blend

In one embodiment, the molding blend may have a Brix number about 84 before forming the mold step. In one embodiment, the molding blend has a Brix number from about 82 to about 87 before forming the mold step.

In one embodiment, the molding blend comprises gelatin at a concentration from about 7% to about 10% by weight. In one embodiment, the molding blend comprises gelatin at a concentration of about 9%. In one embodiment, the molding blend is a flowable liquid. In one embodiment, the molding blend has a viscosity of from about 250 cP to about 6000 cP at at an elevated temperature. In one embodiment, the elevated temperature is from 120° F. to about 180° F., from about 145° F. to about 150° F., from about 140° F. to about 150° F.

In one embodiment, the forming the molding blend comprises injecting the molding blend into a mold and forming the molding blend into a gummy composition dosage or a chewable semi-solid pharmaceutical dosage. In one embodiment the molding blend sets into a gummy composition dosage in less than 15 minutes. In one embodiment, the molding blend sets into a gummy composition dosage in less than 5 minutes. In one embodiment, the molding blend sets into a gummy composition dosage in about 3 minutes.

In one embodiment, the gummy composition dosage has a weight from about 2 grams to about 8 grams. In one embodiment, the gummy composition dosage has a weight from about 3 grams to about 5 grams. In one embodiment, the gummy composition dosage has a weight of about 5 grams. In one embodiment, the gummy composition dosage has a weight of about 7.5 grams.

In one embodiment, the method comprises providing a gelling blend comprising gelatin, optionally a plasticizing agent, and water; providing an active blend comprising an active agent; providing a combining the gelling blend with the active blend to yield a matrix blend; combining the matrix blend with a bonding blend to provide a molding blend; and forming the molding blend into individual gummy composition dosages. The gelling blend may have a residual moisture content to between 5% by weight to 25% by weight.

The gummy composition dosage may be any shape. In one embodiment, the gummy dosage is hexagon shaped. In one embodiment, the gummy dosage is square or cube shaped. In one embodiment, the gummy dosage is gum drop shaped. Example shapes may include without limitation half ball shaped, disk shaped, tablet shaped, heart shaped, flower shaped, letter shaped, toy shaped, animal shaped, or figurine shape.

Gummy Composition and Active Agent

In one aspect, the disclosure provides gelatin based gummy composition that contains an active agent. The active agent could be a nutraceutical agent or a pharmaceutical agent. The active agent may represent from about 1% to about 40% weight of the gummy composition. In one embodiment, the active agent represent from about 2% to about 25% weight of the gummy composition. In one embodiment, the active agent represent from about 5% to about 10% weight of the gummy composition. In one embodiment, the active agent represent from about 2% to about 5% weight of the gummy composition.

In one embodiment, the gummy composition is a dietary or nutritional supplement. In one embodiment, the active agent may be an herbal extract, an herb powder, a natural product, a vitamin, an amino acid, a lipid, a fatty acid, a mineral, a co-enzyme, a hormone, an antioxidant.

The active agent may be or a pharmaceutical agent or an active pharmaceutical ingredient (API). The pharmaceutical agent may be small molecule compound or a biologics. The pharmaceutical agent may be hydrophilic or lipophilic. In one embodiment, the active agent comprises large, hydrophilic and unstable proteins, oligonucleotides and polysaccharides, as well as conventional small drug molecules.

In one embodiment, the disclosure provides gelatin based gummy pharmaceutical composition. In one embodiment, the gummy composition is an OTC drug. In one embodiment, the gummy composition is a legend drug. In one embodiment, the gummy composition is a prescription drug.

In one embodiment, the active agent may be an antibacterial agent, an antifungal agent, a pain or fever reliever, an analgesics, an anti-inflammatory agent, a steroid, a diabetic medication, an antidiarrheal agent, an antiulcer agent, a proton pump inhibitor, a laxative or cathartic agent, a diuretic agent, an antacid, an antihistamine, a decongestant, an antitussive or expectorant agent, a cough suppressant, a cold medicine, a motion sickness medication, a medication for treating alcoholism or opioid dependence, a smoke cessation agent, an anti-anxiety agent, an antidepressant, a mood stabilizer, an antipsychotic agent, a stimulant, a benzodiazepine, an anxiolytic agent, a Z-drug, a melatonergic agent, a barbiturate, an antidepressant, an antipsychotic agent, a cardiovascular agent, an anti-cancer agent, an immune suppressant, a blood thinner, or a combination thereof.

Representative antibacterial agent may include without limitation a penicillin, a cephalosporin, a macrolide, a fluoroquinolone, a sulfonamide, a tetracycline, an aminoglycoside, or a combination thereof.

Representative antibacterial agent may include without limitation bacitracin, clotrimazole, miconazole, penicillin, amoxicillin, cephalexin, erythromycin, clarithromycin, azithromycin, ciprofloxacin, levofloxacin, ofloxacin, co-trimoxazole, trimethoprim, tetracycline, doxycycline, gentamicin, tobramycin, or a combination thereof.

Representative pain or fever reliever include without limitation acetaminophen, aspirin, salicylic acid, ibuprofen, naproxen, or a combination thereof.

Representative antidiarrheal agent include without limitation loperamide, atropine, bismuth subsalicylate, Lactobacillus acidophilus, difenoxin, diphenoxylate, attapulgite or a combination thereof.

Representative antiulcer agent includes without limitation cimetidine, famotidine, nizatidine, ranitidine, misoprostol, sucralfate or a combination thereof.

Representative proton pump inhibitor include without limitation omeprazole, esomeprazole, rabeprazole, pantoprazole, lansoprazole, dexlansoprazole or a combination thereof.

Representative laxative or cathartic agent include without limitation bisacodyl, docusate, MiraLAX, psyllium, senokot, castor oil, magnesium hydroxide, magnesium citrate, magnesium sulfate, lactulose or a combination thereof.

Representative antihistamine include without limitation cetirizine, diphenhydramine, loratadine, chlorpheniramine, brompheniramine, alimemazine, cyprohetadine, doxylamine, hydroxyzine, promethazine, or a combination thereof.

Representative decongestant include without limitation pseudoephedrine, oxymetazoline, phenylephrine or a combination thereof.

Representative antitussive and expectorant include without limitation guaifenesin, codeine phosphate, dextromethorphan hydrobromide, or a combination thereof.

Representative cough suppressant include without limitation dextromethorphan.

Representative medication for treating alcoholism or opioid dependence include without limitation acamprosate, baclofen, buprenorphine, naloxone, clonidine, disulfiram, methadone, naltrexone, ondansetron, or a combination thereof.

Representative smoke cessation agent include without limitation s nicotine, bupropion, cytosine, varenicline, or a combination thereof.

Representative mood stabilizer include without limitation carbamazepine, gabapentin, lamotrigine, levetiracetam, lithium salt, oxcarbazepine, topiramate, sodium valproate, divalproex sodium, valproic acid,

Representative antipsychotic agent include without limitation aripiprazole, asenapine, chlorpromazine, olanzapine, paliperidone, quetiapine, risperidone, ziprasidone,

Representative stimulant include without limitation amphetamine, dexamfetamine, lisdexamfetamine, methylphenidate, dexmethylphenidate, metamfetamine, or a combination thereof.

Representative benzodiazepine include without limitation alprazolam, bromazepam, chlordiazepoxide, clobazam, clonazepam, clorazepate, diazepam, lorazepam, oxazepam, tofisopam, brotizolam, estazolam, flunitrazepam, flurazepam, loprazolam, lormetazepam, midazolam, nimetazepam, nitrazepam, phenazepam, quazepam, temazepam, triazolam, or a combination thereof.

Representative anxiolytic agent includes without limitation buspirone, hydroxyzine, meprobamate, pregabalin, or a combination thereof.

Representative Z-drug include without limitation eszopicolone, zaleplon, zolpidem, zopiclone, or a combination thereof.

Representative melatonergic agent include without limitation agomelatine, ramelteon, or a combination thereof.

Representative barbiturate include without limitation amobarbital, secobarbital, butobarbital, cyclobarbital, diazepam, pentobarbital, phenobarbital, secobarbital, or a combination thereof.

Representative antidepressant include without limitation citalopram, clomipramine, dexopin, escitalopram, fluoxetine, fluvoxamine, imipramine, mirtazapine, paroxetine, sertraline, trazodone, amitriptyline, doxepin, mianserin, mirtazapine, trazodone, trimipramine, or a combination thereof.

Representative antipsychotic agent include without limitation chlorprothizene, levomepromazine, perazine, promethazine, prothipendyl, sulpiride, thioridazine, zuclopenthixol, perphenazine, beneperidol, bromperidol, fluphenazine, fluspirilen, haloperidol, pimozide, amisulpride, aripiprazole, asenapine, chloropromazine, clozapine, flupenthixol, lloperidone, melperone, olanzapine, paliperidone, penfluridol, quetiapine, reserpine, risperidone, sertindole, thiothizene, trifluoperazine, zipraisdone, zotepine, pericyazine, carbamazepine, valproic acid, or a combination thereof.

Representative cardiovascular agent include without limitation an aldosterone receptor antagonist, an angiotensin converting enzyme inhibitor, an angiotensin receptor blocker, a neprilysin inhibitor, an antiadrenergic agent, an antianginal agent, an antiarrhythmic agent, an anticholinergic chronotropic agent, an antihypertension agent, an ACE inhibitor, an angiotensin II inhibitor, an antiadrenergic agent, a beta blocker, a diuretic agent, a beta-adrenergic blocker, a calcium channel blocker, a catecholamine, an inotropic agent, a vasodilator, a renin inhibitor, a sclerosing agent, a vasopressin antagonist, a vasopressor, an anti-cholesterol agent, or a combination thereof.

Representative antihypertension agent include without limitation diazoxide, fenoldopam, nitroprusside, a thiazide, or a combination thereof.

Representative active ingredient include without limitation aspirin, acetaminophen, ibuprofen, diphenhydramine, or a derivative thereof.

In one embodiment, the gummy dosage comprises from about 1 mg to about 500 mg or from about 1 mg to about 1 gram of the active agent. In one embodiment, the gummy pharmaceutical dosage comprises about 81 mg of aspirin or its derivative. In one embodiment, the gummy pharmaceutical dosage comprises about 325 mg of aspirin or its derivative. In one embodiment, the gummy pharmaceutical dosage comprises about 2 mg, 5 mg, 10 mg, 20 mg, 30 mg, 40 mg, or 100 mg of cetirizine or its derivative. In one embodiment, the gummy pharmaceutical dosage comprises about 2 mg, 5 mg, 10 mg, 20 mg, 30 mg, 40 mg, 50 mg, or 100 mg of loratadine or its derivative. In one embodiment, the gummy pharmaceutical dosage comprises about 6 mg, 10 mg, 12 mg, 24 mg, or 48 mg of diphenhydramine or its derivative. In one embodiment, the gummy pharmaceutical dosage comprises about 25 mg, 50 mg, 100 mg, 200 mg, or 400 mg of ibuprofen or its derivative. In one embodiment, the gummy pharmaceutical dosage comprises about 65 mg, 125 mg, 250 mg, or 500 mg of acetaminophen or its derivative. In one embodiment, the gummy dosage comprises a PDE5 inhibitor. In one embodiment, the gummy pharmaceutical dosage comprises about 2 mg, 5 mg, 10 mg, 20 mg, 30 mg, 40 mg, or 100 mg of PDE5 inhibitor or its derivative. Example PDE5 inhibitors include without limitation avanafil, lodenafil, mirodenafil, sildenafil, tadalafil, vardenafil, udenafil, zaprinast, icariin, benzamidenafil, dasantafil, or its derivative thereof.

In one embodiment, the active agent is acidic. In one embodiment, the active agent comprises at least one functional group having a pKa not exceeding 9. In one embodiment, the active agent comprises at least one functional group having a pKa not exceeding 6. In one embodiment, the active agent comprises at least one functional group having a pKa from about 3 to about 6. In one embodiment, the active agent comprises at least one functional group having a pKa from about 2 to about 9. In one embodiment, the active agent comprises at least one functional group having a pKa from about 3 to about 5.

In one embodiment, the functional group comprises acidic functional groups. In one embodiment, the functional group comprises a carboxylic group. In one embodiment, the active agent comprises a phenol group. In one embodiment, the active agent comprises aspirin, salicylic acid, penicillin, fenoprofen, moxifloxacin, cromolyn salt, or a derivative thereof. In one embodiment, the active agent comprises sulfamethoxazole, phenol barbital, phenytoin, chlorthalidone, vildagliptin, phenobarbital, furosemide, tetracycline, or a derivative thereof. In one embodiment, the active agent comprises aspirin and caffeine. In one embodiment, the aspirin and caffeine has a molar ratio from about 15:1 to about 100:1.

Method of Using

In one aspect, the disclosure provides methods and compositions for delivering an active agent through oral mucosal delivery route. The oral mucosal drug delivery may have significant advantage of conventional routes of administration. See, for example, A Detailed Review on Oral Mucosal Drug Delivery System, International Journal of Pharmaceutical Sciences and Research, ICV (2015): 90.24; ISSN (Online): 0975-8232, ISSN (Print): 2320-5148, which is incorporated herein by reference in its entirety.

Oral mucosal drug delivery is an alternative method of systemic drug delivery that offers several advantages over both injectable and enteral methods. Because the oral mucosa is highly vascularized, drugs that are absorbed through the oral mucosa directly enter the systemic circulation, bypassing the gastrointestinal tract and first-pass metabolism in the liver. For some drugs, this results in rapid onset of action via a more comfortable and convenient delivery route than the intravenous route.

The disclosure provides gummy compositions and dosages that are configured to be consumed by chewing therefore delivering the active agent through oral mucosal absorption. Through extensive research and by adjusting the ratio between the gelatin blend, the bonding blend, and optionally plasticizing blend, as well as the MW of the gelling and/or bonding blend, the gummy composition with controlled melting and chewing time can be achieved. In one embodiment, the gummy composition is configured to melt in an oral cavity therefore provide a chewing time not less than 20 seconds, 30 seconds, 1, 2, 3, 4, 5, 6, 7, 8, 9, or 10 minutes. In one embodiment, the gummy composition is gum like and may provide a chewing time of not less than 5, 7 or 10 minutes.

Examples cardiovascular drugs that can be administered trans-mucosally through the disclosed gummy formulation include nitroglycerin, captopril, verapamil and propafenone, has proven promising. Examples of transmucosal delivery of analgesics using the present gummy formulation may include fentanyl, which may be deliver rapid analgesia for breakthrough pain, providing patients with a noninvasive, easy to use and nonintimidating option. Examples of oral mucosal delivery of sedatives using the present gummy formulation may include midazolam, triazolam and etomidate. Examples of oral mucosal delivery of the anti-nausea drugs using the present gummy formulation may include scopolamine and prochlorperazine. Examples of oral mucosal delivery of drugs may also include compounds for treating erectile dysfunction. Oral transmucosal formulations of testosterone and estrogen have been developed. In clinical studies, sublingual testosterone has been shown to result in increases in lean muscle mass and muscle strength, improvement in positive mood parameters, and increases in genital responsiveness in women. Short-term administration of estrogen to menopausal women with cardiovascular disease has been shown to produce coronary and peripheral vasodilation, reduction of vascular resistance and improvement in endothelial function.

The small molecule compound may be sufficiently small whereby the compound may pass the oral mucosal membrane and enter the blood stream during the gummy chewing process. In some embodiments, the molecule weight of the active compound is from about 50 Dalton to about 500 Dalton. In some embodiment, the molecule weight is from about 100 Dalton to about 250 Dalton. The small molecule compound may be an acidic compound, a basic compound, or a zwitterionic compound.

In one embodiment, the active agent may be asenapine, chlorpheniramine maleate, phenylephrine hydrochloride, guaifenesin, dextromethorphan hydrobromide, loratadine, diphenhydramine, or a combination thereof. In one embodiment, the active agent useful for treating gastrointestinal disorders may be an antacid, an anti-foaming agent, a histamine H2-receptor antagonist (commonly known as a H2 antagonist), proton pump inhibitor, or a combination thereof. Other active agents commonly present in OTC medications or legend medications are suitable for use in the present disclosure.

In one aspect, the disclosure provides methods for using the gummy formation for treating diseases or supplementing diet. In one embodiment, the method includes administering to a subject an effective amount of gummy dosage composition to modulate, control, reduce or treat a disease.

The gummy composition dosage is useful for administration to individuals, including both adults and children as dietary supplement or pharmaceuticals for treating symptoms including without limitation allergies, motion sickness, colds, coughs, pains, fever, gastrointestinal disorders, sleep, neurological disorders, cardiovascular disorders, and lung diseases.

The gummy composition dosage may further include absorption enhancer to assist in the absorption of the active ingredient across the mucosal membrane. The absorption enhance include without limitation bile salts, fatty acids and their salts and esters, azones, surfactants, complexing agents, co-solvents, Lecithin (phosphatidylcholine), chitosan, or chitosan glutamate.

In one embodiment, the gummy composition may further include a flavoring agent, a coloring agent, a bitterness masking agent, or a combination thereof.

In a further aspect, the application provides medication kits including a gummy pharmaceutical composition as disclosed herein. In one embodiment, the kid further includes an instruction for using such gummy composition.

Other embodiments, characteristics, and advantages of the disclosure are apparent after reading the descriptions and examples that follow.

### DETAILED DESCRIPTION

The gummy composition dosage of the disclosure is intended to be chewed by a person such that it is broken up into smaller parts or melt within the oral cavity causing the mucosal absorption of the active agent in the gummy composition. The active agent may be released from the gummy dosage and absorbed efficiently through mucosal membrane into blood stream allowing a quick release and fast absorption of the active ingredient. In addition, the smaller pieces may be easily swallowed thereby providing an administration advantage over traditional tablets based composition dosages.

The gummy composition dosage may have a sufficiently high viscosity that it is not pourable and further does not flow or conform to its container at room temperature. Typically, the gummy composition dosage does not flow at low shear stress and generally exhibits plastic flow behavior. In general, the consistency of the gummy composition dosage is the same as or similar to gelatin-based or pectin-based products.

The composition dosage can have any size and shape such that it can be administered orally and chewed by a person. The person should be able to readily break apart the composition dosage by chewing and swallow the composition dosage without the need for an external source of liquid. Typically, the composition dosage has a length of about 1 cm to about 5 cm, width of about 1 cm to about 5 cm and a height of about 1 cm to about 5 cm. Suitable shapes include, for example, ovals, spheres, cylinders, hemisphere, hexagon, rectangular boxes, and cubes. The composition dosage may be formed into unique shapes and figures including, for example, animals for administration to children (e.g., under the age of 13) and/or adults.

Generally, each individual composition dosage has a total weight of at least 100 mg. In one embodiment, each composition dosage has a total weight of from about 1 g to about 20 g. In one embodiment, each composition dosage has a total weight of from about 1 g to about 15 g. In one embodiment, each composition dosage has a total weight of from about 1 g to about 10 g, for example, about 1 g to about 1.5 g, about 1.5 g to about 2 g, about 2 g to about 2.5 g, about 2.5 g to about 3 g, about 3.5 g to about 4 g, about 4 g to about 4.5 g, about 4.5 g to about 5 g, about 5 g to about 5.5 g, about 5.5 g to about 6 g, about 6 g to about 6.5 g, about 6.5 g to about 7 g, about 7 g to about 7.5 g, about 7.5 g to about 8 g, about 8 g to about 8.5 g, about 8.5 g to about 9 g, about 9 g to about 9.5 g, and about 9.5 g to about 10 g. In one embodiment, each composition dosage has a total weight of about 5 g.

Active agents that are suitable for use in the gummy composition dosage for the disclosure include, by way of example, anti-allergy, antihistamines, antitussives, decongestants, expectorants, anti-cold/flu, analgesics, anti-inflammatories, sleep medications, anti-heartburn medications, anti-gas medications, anti-GERD medications, anti-diarrheas, laxatives, anti-smoking and/or motion sickness medications. In some embodiments, suitable active agents may treat and/or prevent gastrointestinal disorders including, for example, antacids, anti-foaming agents, H2 antagonists, proton pump inhibitors, anti-diarrheas, laxatives, or a combination thereof. Suitable active agents useful in the gummy composition dosage of the disclosure are typically available as over-the-counter medications.

The gummy dosage of the disclosure is further useful for administration to individuals, including both adults and children, to treat and/or prevent allergies, colds and coughs, as well as symptoms of these conditions. Additionally, the gummy composition dosage of the disclosure may be used to treat or prevent gastrointestinal disorders and symptoms thereof such as dyspepsia, peptic ulcer, gastroesophageal reflux disease, upset stomach, heartburn, excessive gas, and the like along with symptoms of these disorders.

In other embodiments, the gummy composition dosage of the disclosure includes one or more active agents useful for the treatment of gastrointestinal disorders and symptoms thereof. Such active ingredients are typically available as over-the-counter medications.

Any suitable active agents may be used in the gummy composition dosage of the present disclosure to treat or prevent one or more symptoms. Example active agents include without limitation, anti-allergy agents such as loratadine, diphenhydramine, cetirizine and fexofenadine; anti-cold or flu agents such as pseudoephedrine, phenylephrine and chlorpheniramine; analgesics such as ibuprofen, aspirin, naproxen, acetaminophen, and codeine; cough medications such as guaifenesin and dextromethorphan; sleep medications such as diphenhydramine and doxylamine; heartburn medications such as ranitidine, cimetidine, famotidine, omeprazole, esomeprazole, lansoprazole, calcium carbonate and bismuth subsalicylate; anti-diarrheas such as loperamide; anti-gas agents such as simethicone; laxatives such as bisacodyl; smoking cessation agents such as nicotine; motion sickness medications such as dimenhydrinate meclizine.

Combinations of two or more active agents may be used in the gummy composition dosage of the disclosure to treat or prevent one or more symptoms. Suitable combinations of active agents include for example: anti-allergy combinations such as loratadine/pseudoephedrine, diphenhydramine/phenylephrine, diphenhydramine/pseudoephedrine, cetirizine/pseudoephedrine, and fexofenadine/pseudoephedrine; anti-cold and flu combinations such as chlorpheniramine/phenylephrine, ibuprofen/phenylephrine, dextromethorphan/ibuprofen/phenylephrine, dextromethorphan/acetaminophen/phenylephrine, acetaminophen/phenylephrine, acetaminophen/pseudoephedrine, doxylamine/dextromethorphan/acetaminophen, acetaminophen/dextromethorphan/guaifenesin/phenylephrine; analgesics combinations such as ibuprofen/caffeine, aspirin/caffeine, naproxen/caffeine, acetaminophen/caffeine, diphenhydramine/ibuprofen, diphenhydramine/acetaminophen, diphenhydramine/naproxen, acetaminophen/caffeine/aspirin; and antitussive combinations such as guaifenesin/dextromethorphan; sleep medication combinations such as diphenhydramine/ibuprofen, diphenhydramine/aspirin, diphenhydramine/naproxen, diphenhydramine/acetaminophen; anti-gas combination such as simethicone/calcium carbonate.

The active agents used in the gummy composition dosage of the disclosure include chlorpheniramine maleate, phenylephrine hydrochloride, guaifenesin, dextromethorphan hydrobromide, loratadine, diphenhydramine, or a combination thereof. In one embodiment, the composition dosage contains a combination of chlorpheniramine maleate and phenylephrine hydrochloride. In another embodiment, the composition dosage contains a combination of dextromethorphan hydrobromide and phenylephrine hydrochloride. Chlorpheniramine maleate is a pharmaceutically acceptable salt of chlorpheniramine.

In some embodiments, the amount of chlorpheniramine maleate present in each composition dosage is from about 0.1 mg to about 30 mg. The amount of chlorpheniramine maleate present in each composition dosage is from about 1 mg to about 10 mg. The amount of chlorpheniramine maleate present in each composition dosage is about 1 mg to about 5 mg. The amount of chlorpheniramine present is about 2 mg or about 4 mg in each composition dosage that has a total weight of about 5 g.

Chlorpheniramine maleate may be present in the composition dosage in amount from about 0.01% by weight to about 1.0% by weight, and about 0.02% to about 0.2% by weight. For an adult dose, chlorpheniramine maleate is present in an amount from about 0.06% by weight to about 0.1% by weight. For a pediatric dose (e.g., children under 13), chlorpheniramine maleate is present in an amount from about 0.03% by weight to about 0.05% by weight.

In some embodiments, the amount of phenylephrine hydrochloride present in each composition dosage is from about 0.1 mg to about 20 mg. The amount of phenylephrine hydrochloride present is from about 2 mg to about 15 mg. The amount of phenylephrine hydrochloride present is from about 3 mg to about 12 mg. The amount of phenylephrine hydrochloride present is about 5 mg or about 10 mg in each composition dosage that has a total weight of about 5 g.

Phenylephrine hydrochloride may be present in the composition dosage in amount from about 0.01% by weight to about 1% by weight, and 0.01% to about 0.5% by weight. For an adult dose, phenylephrine hydrochloride is present in an amount from about 0.15% by weight to about 0.25% by weight. For a pediatric dose (e.g., children under 13), phenylephrine hydrochloride is present in an amount from about 0.05% by weight to about 0.15% by weight.

In some embodiments, the amount of guaifenesin present in each composition dosage is from about 10 mg to about 1,500 mg. The amount of guaifenesin present in each composition dosage is from about 200 mg to about 1,200 mg. The amount of guaifenesin present in each composition dosage is about 100 mg to about 400 mg. The amount of guaifenesin present is about 100 mg, 200 mg or about 400 mg in each composition dosage that has a total weight of about 5 g.

Guaifenesin may be present in the composition dosage in amount from about 0.1% by weight to about 20% by weight, and about 0.5% to about 10% by weight. For an adult dose, guaifenesin is present in an amount from about 0.5% by weight to about 5% by weight. For a pediatric dose (e.g., children under 13), guaifenesin is present in an amount from about 0.1% by weight to about 4% by weight.

In some embodiments, the amount of dextromethorphan hydrobromide present in each composition dosage is from about 1 mg to about 100 mg. The amount of guaifenesin present in each composition dosage is from about 5 mg to about 60 mg. The amount of guaifenesin present in each composition dosage is about 10 mg to about 30 mg. The amount of guaifenesin present is about 10 mg or about 20 mg in each composition dosage that has a total weight of about 5 g.

Dextromethorphan hydrobromide may be present in the composition dosage in amount from about 0.01% by weight to about 2% by weight, and about 0.1% to about 1% by weight. For an adult dose, guaifenesin is present in an amount from about 0.1% by weight to about 1% by weight. For a pediatric dose (e.g., children under 13), guaifenesin is present in an amount from about 0.1% by weight to about 0.8% by weight.

In some embodiments, the amount of loratadine present in each composition dosage is from 1 mg to about 100 mg. The amount of loratadine present in each composition dosage is from about 5 mg to about 50 mg. The amount of loratadine present in each composition dosage is from about 10 mg to about 30 mg. The amount of loratadine present is about 10 mg in each composition dosage that has a total weight of about 5 g.

Loratadine may be present in the composition dosage in amount from about 0.01% by weight to about 2% by weight, and about 0.1% to about 1% by weight. For an adult dose, loratadine is present in an amount from about 0.1% by weight to about 1% by weight. For a pediatric dose (e.g., children under 13), loratadine is present in an amount from about 0.1% by weight to about 0.5% by weight.

In some embodiments, the amount of diphenhydramine hydrochloride present in each composition dosage is from 1 mg to about 100 mg. The amount of diphenhydramine hydrochloride present in each composition dosage is from about 5 mg to about 50 mg. The amount of diphenhydramine hydrochloride present in each composition dosage is from about 10 mg to about 30 mg. The amount of diphenhydramine hydrochloride present is about 12.5 mg or 25 mg.

Diphenhydramine hydrochloride may be present in the composition dosage in amount from about 0.01% by weight to about 2% by weight, and about 0.1% to about 1% by weight. For an adult dose, diphenhydramine hydrochloride is present in an amount from about 0.1% by weight to about 1% by weight. For a pediatric dose (e.g., children under 13), diphenhydramine hydrochloride is present in an amount from about 0.1% by weight to about 0.5% by weight.

Chlorpheniramine maleate and phenylephrine hydrochloride may optionally both be present in combination in the composition dosage. In such embodiments, chlorpheniramine maleate is present in an amount of about 2 mg and phenylephrine hydrochloride is present in an amount of about 5 mg. In one embodiment, chlorpheniramine maleate is present in an amount of about 4 mg and phenylephrine hydrochloride is present in an amount of about 10 mg. Typically, a pediatric dose contains about 2 mg chlorpheniramine maleate and 5 mg phenylephrine hydrochloride and an adult dose contains about 4 mg chlorpheniramine maleate and 10 mg phenylephrine hydrochloride.

In embodiments, dextromethorphan hydrobromide and phenylephrine hydrochloride are both present in the composition dosage. Dextromethorphan hydrobromide is present in an amount of about 10 mg and phenylephrine hydrochloride is present in an amount of about 5 mg. Dextromethorphan hydrobromide is present in an amount of about 20 mg and phenylephrine hydrochloride is present in an amount of about 10 mg.

In other embodiments, the active agent is an antacid, anti-foaming agent, histamine H2-antagonist, proton pump inhibitor, anti-diarrheal, laxative, antibiotics, anti-fungal, anti-diabetes, anti-cancer, or combination thereof, that are useful for the treatment and/or prevention of gastrointestinal disorders or symptoms thereof.

Suitable antacids including, but are not limited to, potassium bicarbonate, sodium bicarbonate, calcium bicarbonate, aluminum bicarbonate, magnesium bicarbonate, magnesium hydroxide, calcium carbonate, aluminum hydroxide, and combinations thereof.

In one embodiment, the antacid is calcium carbonate. Calcium carbonate has the formula Ca2CO3. The calcium carbonate can be anhydrous calcium carbonate or a hydrate thereof.

Suitable histamine H2-receptor antagonists include, but are not limited to, cimetidine, ranitidine, famotidine, and nizatidine. In one embodiment, the histamine H2-receptor antagonist is famotidine. Suitable proton pump inhibitors include, but are not limited to, omeprazole, lansoprazole, dexlansoprazole, esomeprazole, pantoprazole, rabeprazole, and ilaprazole. In an embodiment, the proton pump inhibitor is omeprazole. A suitable anti-diarrheal includes, but is not limited to, loperamide. A suitable laxative includes, but is not limited to, bisacodyl. A suitable anti-gas agent includes, but is not limited to, simethicone.

The amount of active agent in the gummy composition dosage will vary for each different active depending on its use. The amount present will usually be less for gummy composition dosages that are to be administered to children. For gummy composition dosages for use with gastrointestinal disorders, the active ingredient may be present in an amount sufficient to treat and/or prevent gastrointestinal disorders and symptoms thereof (e.g., bloating, discomfort and/or pain) including, for example, dyspepsia, peptic ulcer, gastroesophageal reflux disease, upset stomach, heartburn, and excessive gas. Typically, the gummy composition dosage contains about 0.1 mg to 1 g of the active agent. In one embodiment, the gummy composition dosage contains one or more active agents in an amount from about 0.01% by weight to about 10% by weight.

The gummy composition dosage of the disclosure may be administered once per day or multiple times per day to provide relief for various symptoms affecting an individual. For example, chlorpheniramine maleate may be administered to treat symptoms of allergic rhinitis or sinusitis. Phenylephrine hydrochloride may be administered to treat symptoms of nasal congestion. Typical dosing of chlorpheniramine maleate for adults is 4 mg every 4-6 hours and for children (i.e., 6-11 years old) is 2 mg every 4-6 hours. Typical dosing of phenylephrine hydrochloride for adults is 10 mg every 4-6 hours and for children (i.e., 6-11 years old) is 5 mg every 4-6 hours.

Aspirin may be administered to treat symptoms of fever, pain or for cardiovascular health. Typical dosing of aspirin for adults is 81 mg for cardiovascular health and 325 mg for pain or fever relief and for children (i.e., 6-11 years old) is 81 mg.

Naproxen may be administered to relieve pain. Typical dosing of naproxen for adults is 275 mg to 500 mg and for children (i.e., 6-11 years old) is 250 mg or less.

Acetaminophen may be administered to relieve pain or fever. Typical dosing of acetaminophen for adults is 325 mg to 650 mg and for children (i.e., 6-11 years old) is 10-15 mg/Kg.

Guaifenesin may be administered to treat symptoms of congestion in the chest and throat. Typical dosing of guaifenesin for adults is 200 mg to 400 mg every 4-6 hours and for children (i.e., 6-11 years old) is 100 mg to 200 mg every 4-6 hours.

Dextromethorphan hydrobromide may be administered to treat symptoms of a cough. Typical dosing of dextromethorphan hydrobromide for adults is 10 mg to 30 mg every 4-8 hours and for children (i.e., 6-11 years old) is 5 mg to 10 mg every 4 hours.

Loratadine, cetirizine, or diphenhydramine may be administered to treat symptoms of allergic rhinitis and urticaria. Typical dosing of loratadine for adults and children (i.e., 6-11 years old) is 10 mg per day. Typical dosing of cetirizine for adults and children (i.e., 6-11 years old) is 10 mg per day. Typical dosing of diphenhydramine for adults and children (i.e., 6-11 years old) is 12.5 mg or 25 mg per day.

In embodiments where the active agent is an antacid, the amount of antacid present in each composition dosage is from about 10 mg to about 2 g. The amount of antacid present in each composition dosage is from about 100 mg to about 1 g. The amount of antacid present in each composition dosage is about 500 mg to about 1 mg. The amount of antacid present is about 750 mg or about 800 mg in each composition dosage that has a total weight of about 5 g. The antacid may be present in the composition dosage in amount from about 1% by weight to about 30% by weight, and about 5% to about 20% by weight.

In embodiments where the active agent is an anti-foaming agent (also referred to herein as an anti-gas agent), the amount of anti-foaming agent present in each composition dosage is from about 1 mg to about 500 mg. The amount of anti-foaming agent present is from about 5 mg to about 250 mg. The amount of anti-foaming agent present is from about 10 mg to about 100 mg. The amount of anti-foaming agent present is about 20 mg or about 80 mg in each composition dosage that has a total weight of about 5 g.

The anti-foaming agent may be present in the composition dosage in amount from about 0.01% by weight to about 5% by weight, and 0.1% to about 5% by weight. For an adult dose, the anti-foaming agent is present in an amount from about 0.15% by weight to about 0.25% by weight. For a pediatric dose (e.g., children under 13), the anti-foaming agent is present in an amount from about 0.05% by weight to about 0.15% by weight.

In some embodiments, the active agent is a histamine H2-receptor antagonist. In these embodiments, the amount of histamine H2-receptor antagonist present in each composition dosage is from about 1 mg to about 500 mg., the amount of histamine H2-receptor antagonist is from about 5 mg to about 250 mg. The amount of histamine H2-receptor antagonist present is from about 10 mg to about 100 mg. The amount of histamine H2-receptor antagonist present is about 20 mg or about 80 mg in each composition dosage that has a total weight of about 5 g.

In some embodiments, the active agent is a proton pump inhibitor. In these embodiments, the amount of proton pump inhibitor present in each composition dosage is from about 1 mg to about 500 mg. The amount of proton pump inhibitor is from about 5 mg to about 250 mg. The amount of proton pump inhibitor present is from about 10 mg to about 100 mg. The amount of proton pump inhibitor present is about 20 mg or about 80 mg in each composition dosage that has a total weight of about 5 g.

The gelatin based gummy composition disclosed herein comprises gelatin as the gelling agent. Without being bound by any theory, through extensive testing and research, the applicant discovered that the presence of gelatin not only assists with gelling of the gummy composition dosage but also, through the interaction of various ingredients as well as the methods of making steps disclosed herein, serves to modulate, reduce, or mask the taste of the active ingredients.

Any suitable type of gelatin may be present in the composition dosage. For example, the gelatin may be animal-derived gelatin, chemically-modified gelatin, physically-modified gelatin, and combinations thereof. Animal-derived gelatin may be derived from any suitable source such as, for example, pigskin or bovine bone.

In one embodiment, the gelatin may be hydrolyzed gelatin. Hydrolyzed gelatin is also commonly known as hydrolyzed collagen, collagen hydrolysate, and collagen peptide.

Hydrolyzed gelatin having a molecular weight ranging from about 2,500 to about 5,000 may be used. An example of a suitable hydrolyzed gelatin is Peptiplus® powder from Gelita.

Gelatin may be generally present in the gummy composition dosage in an amount from about 0.01% by weight to about 15% by weight., gelatin is present in an amount of from about 0.5% by weight to about 8% by weight, for example from about 0.5% to about 1%, from about 1% to about 1.5%, from about 1.5% to about 2%, from about 2% to about 2.5%, from about 2.5% to about 3%, from about 3% to about 3.5%, from about 3.5% to about 4%, from about 4% to about 4.5%, from about 4.5% to about 5%, from about 5% to about 5.5%, from about 5.5% to about 6%, from about 6% to about 6.5%, from about 6.5% to about 7%, from about 7% to about 7.5%, and from about 7.5% to about 8%. Gelatin is present in an amount from about 1% by weight to about 5% by weight.

The gelatin based gummy composition dosage disclosed herein may further include a second gelling agent. The second gelling agent may be used to provide the composition dosage with the desired characteristics including, for example, gummy structure, shape and texture. The second gelling agent may be any USP (U.S. Pharmacopeia) listed gelling agents, which is incorporated herein by reference in its entirety.

In one embodiment, the second gelling agent may be pectin. It is known that gelatin based gummy matrix is not stable under the acidic condition and acid disrupts the gelatin gummy matrix. It is further known that pectin needs acidic bonding blend to gel and form the gummy matrix. Through extensive testing and research, the applicant was able to provide gummy composition including two combined gelling agents: gelatin and pectin and methods of making such gummy compositions.

Pectin may be generally present in the gummy composition dosage in an amount of from about 0.01% by weight to about 10% by weight. Pectin is present in an amount of from about 0.5% by weight to about 7% by weight, for example from about 0.5% to about 1%, from about 1% to about 1.5%, from about 1.5% to about 2%, from about 2% to about 2.5%, from about 2.5% to about 3%, from about 3% to about 3.5%, from about 3.5% to about 4%, from about 4% to about 4.5%, from about 4.5% to about 5%, from about 5% to about 5.5%, from about 5.5% to about 6%, from about 6% to about 6.5%, and from about 6.5% to about 7%. Pectin is present in an amount from about 1% by weight to about 5% by weight.

The gummy composition dosage of the disclosure includes a bonding blend. Example bonding blends include varieties of saccharides. Generally, saccharide is present in an amount from about 30% by weight to about 99% by weight of the composition dosage. In one embodiment, saccharide is present in an amount from about 40% by weight to about 95% by weight, for example, from about 40% to about 45%, from about 45% to about 50%, from about 50% to about 55%, from about 55% to about 60%, from about 60% to about 65%, from about 65% to about 70%, from about 70% to about 75%, from about 75% to about 80%, from about 80% to about 85%, from about 85% to about 90%, and from about 90% to about 85%.

In some embodiments, the gummy composition dosage includes a polyol. Polyols are also referred to as sugar alcohols. Without being bound by any theory, the presence of a polyol is believed to promote the stability of the gummy composition dosage of the disclosure.

Suitable polyols include, for example, hydrogenated starch hydrolysates, isomalt, lactitol, maltitol, mannitol, sorbitol, erythritol, and xylitol. Combinations of polyols may be used. In one embodiment, the polyol is hydrolyzed starch hydrolysates (HSH). HSH typically contains substantial quantities of hydrogenated oligo- and poly-saccharides in addition to monomeric and dimeric polyols. HSH is commonly known to include polyglycitol. An example of a commercially available HSH is Hystar® 3375 syrup (75% solids), Hystar® 4075 and Hystar® 6075 supplied by SPI Polyols. Other commercially available HSH include 75/400 from Roquette and Stabilite® liquid HSH and Stabilite® powdered HSH supplied by Corn Products Specialty Ingredients.

One or more polyols may be present in the gummy composition dosage in an amount from about 30% by weight to about 99% by weight. In one embodiment, one or more polyols may be present in an amount from about 40% by weight to about 90% by weight, for example, about 40% to about 50%, about 50% to about 60%, about 60% to about 70%, about 70% to about 80%, and about 80% to about 90%. In one embodiment, one or more polyols may be present in an amount from about 40% by weight to about 60% by weight.

In embodiments in which one or more polyols are present, the ratio of polyol to sugar is typically from about 1:10 to about 10:1 by dry weight., the ratio of polyol to sugar is from about 1:2 to about 2:1 by dry weight, for example, from about 1:1.5 to about 1:5.1.

In other embodiments, the ratio of polyol to gelling agent is from about 40:1 to about 1:1 by dry weight. In one embodiment, the ratio of polyol to gelling agent is from about 30:1 to about 10:1 by dry weight.

In some embodiments, the gummy composition dosage includes corn syrup, tapioca syrup, maple syrup, beet syrup, malt, or any other syrup. The syrup may be present without a polyol. In one embodiment, corn syrup may be present in addition to a polyol. Any suitable corn syrup may be used, for example, corn syrup having 10-65 DE (dextrose equivalents), corn syrup 42-43 DE Corn syrup may contain about 50% by weight to about 90% by weight solids, about 80% solids.

Syrup may be present in the gummy composition dosage in an amount from about 30% by weight to about 99% by weight, corn syrup may be present in an amount from about 40% by weight to about 90% by weight, for example, about 40% to about 50%, about 50% to about 60%, about 60% to about 70%, about 70% to about 80%, and about 80% to about 90%.

In embodiments in which syrup is present, the ratio of syrup to sugar is typically from about 1:10 to about 10:1 by dry weight. In one embodiment, the ratio of the syrup to sugar is from about 1:2 to about 2:1 by dry weight, for example, from about 1:1.5 to about 1:5.1.

In other embodiments, the ratio of the syrup to gelling agent is from about 20:1 to about 1:1 by dry weight. In one embodiment, the ratio of the syrup to gelling agent is from about 10:1 to about 2:1 by dry weight.

The gummy composition dosage may optionally include a pH adjusting agent. Any suitable pH adjusting agent may be used that is sufficient to adjust the pH during the manufacture of the composition dosage to yield the desired pH. By way of example, the pH adjusting agent may be sodium citrate, citric acid, sodium ascorbate and ascorbic acid. Two or more pH adjusting agents may be used. The pH adjusting agent may be supplied in solid form (e.g., as a powder) or in aqueous solution. For example, citric acid may be supplied in a 50% solution. In one embodiment, the pH adjusting agent is sodium citrate or citric acid. Both sodium citrate and citric acid are included in the gummy composition dosage as pH adjusting agents.

The pH adjusting agent may be present in the gummy composition dosage in an amount from about 0.1% by weight to about 5% by weight. In one embodiment, the pH adjusting agent may be present in an amount from about 1% to about 5% by weight, for example, from about 1% to about 1.5%, from about 1.5% to about 2%, from about 2% to about 2.5%, from about 2.5% to about 3%, from about 3% to about 3.5%, from about 3.5% to about 4.0%, from about 4% to about 4.5%, and from about 4.5% to about 5%.

In some embodiments, sodium citrate is present in an amount from about 0.1% by weight to about 1% by weight. In one embodiment, sodium citrate is present in an amount from about 0.1% by weight to about 0.5% by weight, for example, from about 0.1% to about 0.2%, from about 0.2% to about 0.3%, from about 0.3% to about 0.4%, and from about 0.4% to about 0.5%.

In other embodiments, citric acid is present (as 50% aqueous solution) in an amount from about 0.5% by weight to about 3% by weight, for example from about 0.5% to about 1%, from about 1% to about 1.5%, from about 1.5% to about 2%, from about 2% to about 2.5%, and from about 2.5% to about 3%.

In certain embodiments, the gummy composition dosage contains glycerin, also commonly known as glycerol. Without being bound by any theory, glycerin is believed to function as an emollient to stability the composition dosage during its preparation. In one embodiment, glycerin USP is used. In some embodiments, glycerin is present in the gummy composition dosage in addition to the absence of gelatin.

Glycerin may be present in the gummy composition dosage in an amount from about 0.1% by weight to about 10% by weight., glycerin is present in an amount from about 0.5% by weight to about 5% by weight, for example from about 0.5% to about 1%, from about 1% to about 1.5%, from about 1.5% to about 2.0%, from about 2.0% to about 2.5%, from about 2.5% to about 3.0%, from about 3.0% to about 3.5%, from about 3.5% to about 4.0%, from about 4.0% to about 4.5%, and from about 4.5% to about 5.0%.

In some embodiments, the gummy composition dosage contains a flavorant. Any suitable food-grade flavorant may be used to suppress the bitterness of the active ingredients to provide a pleasant taste to the composition dosage upon chewing and swallowing. A mixture of two or more flavorants may be used to yield the desired taste characteristic.

Suitable flavorants include artificial sweeteners such as, for example, sucralose, acesulfame potassium, stevia, sodium saccharine, erythritol, and aspartame. Another suitable flavorant may be a fraction of the lactone group such as, for example, decalactone and dodecalactone (e.g., gamma dodecalactone). Lactone fractions are typically supplied in a propylene glycol solution, in particular from 0.5% to 1% in propylene glycol solution. The flavorant may be orange or cherry flavors. In one embodiment, the flavorant may be menthol.

In one embodiment, the flavorant is an artificial sweetener. In one embodiment, the artificial sweetener is sucralose.

The flavorant may be present in an amount up to about 1% by weight, up to about 0.5% by weight, for example, up to about 0.01%, up to about 0.05%, up to about 0.1%, up to about 0.2%, up to about 0.3%, up to about 0.4%, and up to about 0.5%. In certain embodiments, the amount of flavorant present is in a range bounded by any of the foregoing values. Fractions of the lactone group may be present in an amount of from about 1 ppm to 50 ppm, from about 2 ppm to about 10 ppm, and more from about 3 ppm to about 9 ppm.

A colorant may optionally be added to provide a suitable appearance for the gummy composition dosage. Examples of suitable colorants include red or yellow dyes such as FD&C Red #40 and FD&C Yellow #6. Two or more colorants may be combined.

The gummy composition dosage may have a water content, also referred to as a residual moisture content, of less than about 15% by weight, e.g., about 14% or less, about 13% or less, about 12% or less, about 11% or less, about 10% or less, about 9% or less, about 8% or less, about 7% or less, about 6% or less, or about 5% or less. In other embodiments, the water content of the gummy composition dosage is in a range bounded by any of the foregoing values. In one embodiment, the water content of the gummy composition dosage is from about 8% by weight to about 15% by weight.

In some embodiments, the gummy composition dosage comprises one or more active agents, a gelling agent, gelatin, sugar, a polyol, and a pH adjusting agent. In other embodiments, the gummy composition dosage comprises one or more active agents, a gelling agent, gelatin, sugar, corn syrup, and a pH adjusting agent. In other embodiments, the gummy composition dosage comprises one or more active agents, a gelling agent, sugar, a polyol, glycerin, and a pH adjusting agent.

In some embodiments, the gummy composition dosage comprises one or more active agents, pectin, sugar, hydrolyzed starch hydrolysate, hydrolyzed gelatin, and a pH adjusting agent. In other embodiments, the gummy composition dosage comprises one or more active agents, pectin, sugar, corn syrup, hydrolyzed gelatin, and a pH adjusting agent.

In some embodiments, the gummy composition dosage comprises one or more active agents, pectin, sugar, hydrolyzed starch hydrolysate, glycerin, and a pH adjusting agent.

In some embodiments, the gummy composition dosage comprises: one or more active agents in an amount from about 0.01% by weight to about 10% by weight; pectin in an amount from about 0.5% by weight to about 7% by weight; sugar in an amount from about 40% by weight to about 95% by weight; optionally hydrolyzed starch hydrolysate in an amount from about 40% by weight to about 90% by weight; optionally hydrolyzed gelatin in an amount from about 0.5% by weight to about 8% by weight; sodium citrate in an amount from about 0.1% by weight to about 1% by weight; and citric acid in an amount from about 0.5% by weight to about 3% by weight, wherein the water content of the gummy composition dosage is from about 8% by weight to about 15% by weight.

In some embodiments, the gummy composition dosage comprises: one or more active agents in an amount from about 0.01% by weight to about 10% by weight; pectin in an amount from about 0.5% by weight to about 7% by weight; sugar in an amount from about 40% by weight to about 95% by weight; syrup in an amount from about 40% by weight to about 90% by weight; hydrolyzed gelatin in an amount from about 0.5% by weight to about 8% by weight; sodium citrate in an amount from about 0.1% by weight to about 1% by weight; and citric acid in an amount from about 0.5% by weight to about 3% by weight, wherein the water content of the gummy composition dosage is from about 8% by weight to about 15% by weight.

In some embodiments, the gummy composition dosage comprises: one or more active agents in an amount from about 0.01% by weight to about 10% by weight; pectin in an amount from about 0.5% by weight to about 7% by weight; sugar in an amount from about 40% by weight to about 95% by weight; hydrolyzed starch hydrolysate in an amount from about 40% by weight to about 90% by weight; glycerin in an amount from about 0.1% by weight to about 5% by weight; sodium citrate in an amount from about 0.1% by weight to about 1% by weight; and citric acid in an amount from about 0.5% by weight to about 3% by weight, wherein the water content of the gummy composition dosage is from about 8% by weight to about 15% by weight.

In some embodiments, the gummy composition dosage comprises: chlorpheniramine maleate; phenylephrine hydrochloride; gelatin in an amount from about 2% by weight to about 10% by weight; sugar in an amount from about 40% by weight to about 95% by weight; cyclodextrin in an amount from 0% to 10% by weight, sodium citrate in an amount from about 0.1% by weight to about 1% by weight; and citric acid in an amount from about 0.5% by weight to about 3% by weight, wherein the water content of the gummy composition dosage is from about 8% by weight to about 15% by weight.

In some embodiments, the gummy composition dosage comprises: ibuprofen; gelatin in an amount from about 2% by weight to about 10% by weight; sugar in an amount from about 40% by weight to about 95% by weight; cyclodextrin in an amount from 0% to 10% by weight, sodium citrate in an amount from about 0.1% by weight to about 1% by weight; and citric acid in an amount from about 0.5% by weight to about 3% by weight, wherein the water content of the gummy composition dosage is from about 8% by weight to about 15% by weight.

In some embodiments, the gummy composition dosage comprises: diphenhydramine hydrochloride; gelatin in an amount from about 2% by weight to about 10% by weight; sugar in an amount from about 40% by weight to about 95% by weight; cyclodextrin in an amount from 0% to 10% by weight, sodium citrate in an amount from about 0.1% by weight to about 1% by weight; and citric acid in an amount from about 0.5% by weight to about 3% by weight, wherein the water content of the gummy composition dosage is from about 8% by weight to about 15% by weight.

In some embodiments, the gummy composition dosage comprises: scopolamine; gelatin in an amount from about 2% by weight to about 10% by weight; sugar in an amount from about 40% by weight to about 95% by weight; cyclodextrin in an amount from 0% to 10% by weight, sodium citrate in an amount from about 0.1% by weight to about 1% by weight; and citric acid in an amount from about 0.5% by weight to about 3% by weight, wherein the water content of the gummy composition dosage is from about 8% by weight to about 15% by weight.

In some embodiments, the gummy composition dosage comprises: loratadine; gelatin in an amount from about 2% by weight to about 10% by weight; sugar in an amount from about 40% by weight to about 95% by weight; cyclodextrin in an amount from 0% to 10% by weight, sodium citrate in an amount from about 0.1% by weight to about 1% by weight; and citric acid in an amount from about 0.5% by weight to about 3% by weight, wherein the water content of the gummy composition dosage is from about 8% by weight to about 15% by weight.

In some embodiments, the gummy composition dosage comprises: cetirizine; gelatin in an amount from about 2% by weight to about 10% by weight; sugar in an amount from about 40% by weight to about 95% by weight; cyclodextrin in an amount from 0% to 10% by weight, sodium citrate in an amount from about 0.1% by weight to about 1% by weight; and citric acid in an amount from about 0.5% by weight to about 3% by weight, wherein the water content of the gummy composition dosage is from about 8% by weight to about 15% by weight.

In some embodiments, the gummy composition dosage comprises: aspirin; gelatin in an amount from about 2% by weight to about 10% by weight; sugar in an amount from about 40% by weight to about 95% by weight; cyclodextrin in an amount from 0% to 10% by weight, sodium citrate in an amount from about 0.1% by weight to about 1% by weight; and citric acid in an amount from about 0.5% by weight to about 3% by weight, wherein the water content of the gummy composition dosage is from about 8% by weight to about 15% by weight.

In some embodiments, the gummy composition dosage comprises: hydroxyzine pamoate; gelatin in an amount from about 2% by weight to about 10% by weight; sugar in an amount from about 40% by weight to about 95% by weight; cyclodextrin in an amount from 0% to 10% by weight, sodium citrate in an amount from about 0.1% by weight to about 1% by weight; and citric acid in an amount from about 0.5% by weight to about 3% by weight, wherein the water content of the gummy composition dosage is from about 8% by weight to about 15% by weight.

In some embodiments, the gummy composition dosage comprises: naproxen; gelatin in an amount from about 2% by weight to about 10% by weight; sugar in an amount from about 40% by weight to about 95% by weight; cyclodextrin in an amount from 0% to 10% by weight, sodium citrate in an amount from about 0.1% by weight to about 1% by weight; and citric acid in an amount from about 0.5% by weight to about 3% by weight, wherein the water content of the gummy composition dosage is from about 8% by weight to about 15% by weight.

In some embodiments, the gummy composition dosage comprises: acetaminophen; gelatin in an amount from about 2% by weight to about 10% by weight; sugar in an amount from about 40% by weight to about 95% by weight; cyclodextrin in an amount from 0% to 10% by weight, sodium citrate in an amount from about 0.1% by weight to about 1% by weight; and citric acid in an amount from about 0.5% by weight to about 3% by weight, wherein the water content of the gummy composition dosage is from about 8% by weight to about 15% by weight.

In some embodiments, the gummy composition dosage comprises: Sildenafil Citrate, gelatin in an amount from about 2% by weight to about 10% by weight; sugar in an amount from about 40% by weight to about 95% by weight; cyclodextrin in an amount from 0% to 10% by weight, sodium citrate in an amount from about 0.1% by weight to about 1% by weight; and citric acid in an amount from about 0.5% by weight to about 3% by weight, wherein the water content of the gummy composition dosage is from about 8% by weight to about 15% by weight.

In some embodiments, the gummy composition dosage comprises: Tadalafil; gelatin in an amount from about 2% by weight to about 10% by weight; sugar in an amount from about 40% by weight to about 95% by weight; cyclodextrin in an amount from 0% to 10% by weight, sodium citrate in an amount from about 0.1% by weight to about 1% by weight; and citric acid in an amount from about 0.5% by weight to about 3% by weight, wherein the water content of the gummy composition dosage is from about 8% by weight to about 15% by weight.

In some embodiments, the gummy composition dosage comprises: Vardenafil hydrochloride; gelatin in an amount from about 2% by weight to about 10% by weight; sugar in an amount from about 40% by weight to about 95% by weight; cyclodextrin in an amount from 0% to 10% by weight, sodium citrate in an amount from about 0.1% by weight to about 1% by weight; and citric acid in an amount from about 0.5% by weight to about 3% by weight, wherein the water content of the gummy composition dosage is from about 8% by weight to about 15% by weight.

In some embodiments, the gummy composition dosage comprises: metaformin; gelatin in an amount from about 2% by weight to about 10% by weight; sugar in an amount from about 40% by weight to about 95% by weight; sodium citrate in an amount from about 0.1% by weight to about 1% by weight; and citric acid in an amount from about 0.5% by weight to about 3% by weight, wherein the water content of the gummy composition dosage is from about 8% by weight to about 15% by weight.

In some embodiments, the gummy composition dosage comprises: cannabinoids; gelatin in an amount from about 2% by weight to about 10% by weight; sugar in an amount from about 40% by weight to about 95% by weight; cyclodextrin in an amount from 0% to 10% by weight, sodium citrate in an amount from about 0.1% by weight to about 1% by weight; and citric acid in an amount from about 0.5% by weight to about 3% by weight, wherein the water content of the gummy composition dosage is from about 8% by weight to about 15% by weight.

In some embodiments, the gummy composition dosage comprises: an active agent selected from the group consisting of an antacid, an anti-foaming agent, a histamine H2-receptor antagonist, a proton pump inhibitor, or a combination thereof in an amount from about 0.01% by weight to about 10% by weight; pectin in an amount from about 0.5% by weight to about 7% by weight; sugar in an amount from about 40% by weight to about 95% by weight; hydrolyzed starch hydrolysate in an amount from about 40% by weight to about 90% by weight; hydrolyzed gelatin in an amount from about 0.5% by weight to about 8% by weight; sodium citrate in an amount from about 0.1% by weight to about 1% by weight; and citric acid in an amount from about 0.5% by weight to about 3% by weight, wherein the water content of the gummy composition dosage is from about 8% by weight to about 15% by weight.

In some embodiments, the gummy composition dosage comprises: calcium carbonate, simethicone, famotidine, or omeprazole; pectin in an amount from about 0.5% by weight to about 7% by weight; sugar in an amount from about 40% by weight to about 95% by weight; hydrolyzed starch hydrolysate in an amount from about 40% by weight to about 90% by weight; hydrolyzed gelatin in an amount from about 0.5% by weight to about 8% by weight; sodium citrate in an amount from about 0.1% by weight to about 1% by weight; and citric acid in an amount from about 0.5% by weight to about 3% by weight, wherein the water content of the gummy composition dosage is from about 8% by weight to about 15% by weight.

The gummy composition dosage can be prepared by any suitable method including, for example, a batch process or a continuous process. In some embodiments, the components of the composition dosage are first combined together in a suitable vessel. The components can be combined in any suitable order.

In some embodiments, different blends of components are prepared separately and then combined together to form a final blend from which the gummy composition dosage is obtained. For example, a primary blend may be combined with a secondary blend to form the final blend. A separate blend containing flavorants and/or colorants and an acid solution may optionally be added in the preparation of the final blend.

In one embodiment, a primary blend is prepared by combining pectin, sugar, a polyol, and a pH adjusting agent with water. In one embodiment, the primary blend may be prepared by combining pectin, sugar, corn syrup, and a pH adjusting agent with water. The amount of water and corn syrup. A pH adjusting agent such as, for example, sodium citrate may optionally be added to the primary blend. In some embodiments, the primary blend has a pH from about 2 to about 6, from about 2.5 to about 4, and more from about 2.8 to about 3.8.

In certain aspects, the primary blend is heated at an appropriate temperature and for an appropriate length of time to provide the primary blend with any suitable moisture content for further processing. In one embodiment, the primary blend has a moisture content after cooking from about 5% by weight to about 25% by weight. In one embodiment, the primary blend has a residual moisture content after cooking from about 9% by weight to about 20% by weight, for example, about 9% to about 10%, about 10% to about 11%, about 11% to about 12%, about 12% to about 13%, about 13% to about 14%, and about 14% to about 15%, about 15% to about 16%, about 16% to about 17%, about 17% to about 18%, about 18% to about 19%, and about 19% to about 20%. Generally, the primary blend may be cooked at a temperature of about 230° F. to about 250° F., for example, about 230° F. to about 235° F., about 235° F. to about 240° F., about 240° F. to about 245° F., and about 245° F. to about 250° F.

A secondary blend may be added to the primary blend after cooking is completed. The secondary blend may contain one or more components of the gummy composition dosage. In some embodiments, the secondary blend includes chlorpheniramine maleate, phenylephrine hydrochloride, and hydrolyzed gelatin. In other embodiments, the secondary blend includes calcium carbonate, simethicone, famotidine, or omeprazole, and hydrolyzed gelatin. Water may be added to the secondary blend to dissolve water-soluble components and/or form a homogenous mixture. Other components may be added to the secondary blend including, for example, glycerin, flavorants and colorants. In one embodiment, an additional blend may be prepared containing glycerin, flavorants and colorants. An acid solution may further be prepared containing citric acid to obtain the desired pH of the final blend. The final blend may be obtained by combining the primary blend, secondary blend, additional blend and citric acid in any order.

The final blend may be further processed as needed prior to preparation of the gummy composition dosage. For example, the final blend may be transferred to a depositor hopper having a jacket to maintain a temperature of from about 180° F. to about 210° F., from about 190° F. to about 200° F. After a suitable amount of time, the final blend may be dispensed from the depositor hopper to product the gummy composition dosage of the disclosure.

The gummy composition dosage may be obtained by depositing the final blend into pre-formed molds using conventional techniques. In one embodiment, the molds are blister packs having multiple cavities that provide for unit dose packaging of the gummy composition dosage without having to transfer the composition dosage from a mold to a separate container. The composition dosage solidifies in the molds, which serve as the final packaging. As the temperature of the composition dosage cools, the composition dosage takes its final shape in the cavities of the blister pack. The blister pack is sealed, for example, using foil. One or more blister packs may be packaged in containers. In one embodiment, the composition dosages may be prepared in molds and transferred to other suitable containers.

In one embodiment, a pre-determined amount of the final blend, for example based on weight, is dispensed into each cavity to form individual pieces. The individual pieces contain the desired amount of the active ingredients as described herein. For example, individual pieces may contain 10 mg cetirizine for an adult dose and 5 mg cetirizine for a pediatric dose.

The following examples further illustrate the disclosure but, of course, should not be construed as in any way limiting its scope.

### EXAMPLES

### Example 1. Diphenhydramine Hydrochloride Gummy

This example demonstrates a gummy composition dosage and its method of preparation in accordance with an embodiment of the disclosure. Ingredients include 2.2 g Diphenhydramine HCl, 0.5 g Sodium Benzoate, 0.5 g Potassium Sorbate, 240.0 g Sucrose, 283.0 g Glucose Syrup, 245.0 g Water, 62.4 g Gelatin, 18.8 g Sorbitol, 116.0 g Water, and 10.0 g organic ground Ginger.

The gelatin and sorbitol were shifted together until homogenous using a fork. The dry component mixture was added to 116.0 grams of water rapidly with rapid stirring. The dry mix quickly absorbed the water and became a rubbery mass. The rubbery mass was added to a zip lock bag and heated at 160° F. until free of foam and a clear yellow.

245 grams of water was added to a container and heated to the boiling point. To the boiling water was added diphenhydramine HCl followed by potassium sorbate and sodium benzoate. The components were allowed to dissolve. The water phase was reduced by 100 grams. To the boiling diphenhydramine HCl solution was added glucose syrup with stirring. The solution was then brought back to a boil. Sucrose and ginger were shifted together until homogenous and the mix was added to the diphenhydramine HCl/glucose solution. The solution was heated until 248° F. was reached and 151 grams of water removed.

The solution was then cooled to 200° F. The gelatin solution was slowly added with stirring. The mixture was stirred until homogenous. The solution was then added to silicone molds and the molds were placed in the refrigerator for 90 minutes.

The result was chewy gummy product with 25 mg diphenhydramine HCl per 7.5-gram gummy with good ginger spice flavor.

### Example 2. Diphenhydramine Gummy

Ingredients include: 240.0 g Sucrose, 283.0 g Glucose Syrup, 150.0 g Water, 2.390 g Diphenhydramine Hydrochloride, 62.5 g Gelatin, 18.8 g Sorbitol, 116.0 g Water, 0.5 g Sodium Benzoate, 0.5 g Potassium Sorbate, 1.0 g, Watermelon Flavoring, and 0.5 g Coconut Flavoring

The gelatin and sorbitol were mixed together. The dry component mixture was added to water with dissolved potassium sorbate and sodium benzoate. The dry mix quickly absorbed the water and became a rubbery mass. The rubbery mass was heated at 160° F. until free of foam and a clear yellow.

Water was added to a container followed by the diphenhydramine. The water was brought to a boil and the components were allowed to dissolve. Glucose syrup was added. The sucrose mix was then added to the boiling solution. The solution was heated until a brix level of 85 was reached. The solution was then cooled to 200° F. The gelatin solution was slowly added with stirring. Then the watermelon and coconut flavorings were added. The solution was then added to silicone molds.

### Example 3. Diphenhydramine Gummy

Ingredients include: 240.0 g Sucrose, 200.1 g Ginger Juice Concentrate, 100.0 g Glucose Syrup, 2.388 g Diphenhydramine Hydrochloride, 62.5 g Gelatin, 18.8 g Sorbitol, 0.5 g Sodium Benzoate, and 0.5 g Potassium Sorbate

The gelatin and sorbitol were shifted together. The dry component mixture was added to 116.0 grams of water with dissolved potassium sorbate and sodium benzoate rapidly with rapid stirring. The dry mix quickly absorbed the water and became a rubbery mass. The rubbery mass was heated at 160° F. until free of foam and a clear yellow.

The ginger juice concentrate was added to a container with sucrose and glucose syrup. The components were heated until reflux until a Brix level of 87.5 was reached. The components were cooled to 200° F. and the gelatin mix was added with stirring. The temperature was maintained to remain above 180° F. A mix of 2.388 g of diphenhydramine in 4.035 g water was with stirring. The solution was then added to silicone molds.

### Example 4. Diphenhydramine Gummy

Ingredients include: 240.3 g Sucrose, 200.1 g Ginger Juice Concentrate 100.5 g Glucose Syrup, 2.350 g Diphenhydramine Hydrochloride, 62.5 g Gelatin, 18.8 g Mannitol, 21.2 g Taurine, 0.5 g Sodium Benzoate, and 0.5 g Potassium Sorbate.

The gelatin and mannitol were shifted together until homogenous using a fork. The dry component mixture was added to 116.0 grams of water with dissolved potassium sorbate and sodium benzoate rapidly with rapid stirring. The dry mix quickly absorbed the water and became a rubbery mass. The rubbery mass was heated at 160.F until free of foam and a clear yellow.

The ginger juice concentrate was added to a container with sucrose and glucose syrup. The components were heated until reflux until a Brix level of 87.5 was reached. The components were cooled to 200° F. and the gelatin mix was added with stirring. The temperature was maintained to remain above 180° F. A solution of 2.350 g of diphenhydramine hydrochloride in 6.100 g water was then added. The final Brix was 84. The solution was then added to silicone molds.

### Example 5. Multivitamin and Herbal Ingredient Gummies

Ingredient list: 0.5 g Sodium Benzoate, 0.5 g Potassium Sorbate, 240.3 g Sucrose, 60.0 g Maltodextrin, 0.675 g B-Vitamin/Mineral Premix, 0.675 g Angelica sinensis Root Extract, 208.1 g Glucose Syrup, 245.0 g Water, 62.4 g Gelatin, 18.8 g Sorbitol, 116.0 g Water, 10.0 g Strawberry Extract

The gelatin, vitamin premix, and sorbitol were shifted together until homogeneous. The dry component mixture was added to 116.0 grams of water rapidly with rapid stirring. The dry mix quickly absorbed the water and became a rubbery mass. The rubbery mass was heated at 160 F until free of foam and a clear yellow.

In a separate container were shifted together angelica sinensis root extract, maltodextrin and sucrose. 240.3 grams of water was added to a container and heated. To the water was added potassium sorbate and sodium benzoate. To the boiling water was added glucose syrup. The solution was then brought to a boil. The sucrose mixture was added to the boiling solution. The solution was heated until a Brix of 87-89 has been reached.

The solution was then cooled to 200 F. The extract flavoring was then added. The gelatin solution was then slowly added. The Brix level was 85. The solution was then added to silicone molds. Gelation occurred within 30 minutes. The result was chewy gummy product containing roughly 10 mg of root extract per 7.5 grams gummy with good strawberry flavor and the desired loading of vitamins and minerals.

### Example 6. Multivitamin and Herbal Ingredient Gummies

Ingredient list: 0.5 g Sodium Benzoate, 0.5 g Potassium Sorbate, 240.3 g Sucrose, 60.0 g Maltodextrin, 13.1 g Vitamin—Mineral Premix, 1.000 g Angelica sinensis Root Extract, 208.1 g Glucose Syrup, 245.0 g Water, 62.4 g Gelatin, 18.8 g Sorbitol, 116.0 g Water, 10.0 g Strawberry Extract

The gelatin, vitamin premix, and sorbitol were shifted together until homogeneous. The dry component mixture was added to 116.0 grams of water rapidly with rapid stirring. The dry mix quickly absorbed the water and became a rubbery mass. The rubbery mass was added to a zip lock bag and heated at 160 F until free of foam and a clear yellow.

In a separate container were shifted together angelica sinensis root extract, maltodextrin and sucrose until homogeneous. 240.3 grams of water was added to a container and heated to the boiling point. To the boiling water was added potassium sorbate and sodium benzoate. To the boiling water was added glucose syrup with stirring and allowed to dissolve. The solution was then brought to a boil. The sucrose mixture was them slowly added to the boiling solution. The solution was heated until a Brix of 87-89 has been reached.

The solution was then cooled to 200 F. The extract flavoring was then added with stirring until homogeneous. The gelatin solution was then slowly added with stirring. The mixture was stirred until homogeneous. The Brix level was 85. The solution was then added to silicone molds. Gelation occurred within 30 minutes. The result was chewy gummy product containing roughly 10 mg of root extract per 7.5 grams gummy with good strawberry flavor and the desired loading of vitamins and minerals.

### Example 7. Multivitamin and Herbal Ingredient Gummies

Ingredient list: 240.3 g Sucrose, 60.0 g Maltodextrin, 13.1 g Vitamin—Mineral Premix, 1.000 g Angelica sinensis Root Extract, 208.1 g Glucose Syrup, 245.0 g Water, 62.4 g Gelatin, 18.8 g Sorbitol, 116.0 g Water, 7.5 g Citric Acid, 15.2 g Citric Acid 50% Solution, 10.0 g Strawberry Extract

The gelatin, vitamin premix, and sorbitol were shifted together until homogeneous. The dry component mixture was added to 116.0 grams of water rapidly with rapid stirring. The dry mix quickly absorbed the water and became a rubbery mass. The rubbery mass was added to a zip lock bag and heated at 160 F until free of foam and a clear yellow.

In a separate container were shifted together angelica sinensis root extract, maltodextrin and sucrose until homogeneous.

240.3 grams of water was added to a container and heated to the boiling point. To the boiling water was added glucose syrup with stirring and allowed to dissolve. The solution was then brought to a boil. The sucrose mixture was them slowly added to the boiling solution. The solution was heated until a Brix of 89-90 has been reached. At which time the citric acid was added with stirring and allowed to dissolve. The 50% citric acid solution was then added with stirring. The solution was then cooled to 200 F. The extract flavoring was then added with stirring until homogeneous. The gelatin solution was then slowly added with stirring. The mixture was stirred until homogeneous. The Brix level was 85. The solution was then added to silicone molds and the molds were placed in the refrigerator. Gelation occurred within 30 minutes. The result was chewy gummy product containing roughly 10 mg of root extract per 7.5 grams gummy with good strawberry flavor and the desired loading of vitamins and minerals.

### Example 8. Diphenhydramine Gummy

Ingredients include: 220.0 g Sucrose, 20.0 g beta-cyclodextrin, 200.1 g Ginger Juice Concentrate 100.5 g Glucose Syrup, 2.350 g Diphenhydramine Hydrochloride, 62.5 g Gelatin, 18.8 g Mannitol, 21.2 g Taurine, 0.5 g Sodium Benzoate, and 0.5 g Potassium Sorbate.

The gelatin and mannitol were shifted together until homogenous using a fork. The dry component mixture was added to 116.0 grams of water with dissolved potassium sorbate and sodium benzoate rapidly with rapid stirring. The dry mix quickly absorbed the water and became a rubbery mass. The rubbery mass was added to a zip lock bag and heated at 160° F. until free of foam and a clear yellow.

The ginger juice concentrate was added to a container with sucrose, cyclodextrin and glucose syrup. The components were heated until reflux until a Brix level of 87.5 was reached. The components were cooled to 200° F. and the gelatin mix was added with stirring. The temperature was maintained to remain above 180° F. A solution of 2.350 g of diphenhydramine hydrochloride in 6.100 g water was then added dropwise over a period of 30 seconds with stirring. The final Brix was 84. The solution was then added to silicone molds and the molds were placed in the refrigerator for 90 minutes.

### Example 9. Tadalafil Gummy

Ingredients include: 220.0 g Sucrose, 20.0 g beta-cyclodextrin, 200.0 g Passion Juice Concentrate 100.4 g Glucose Syrup, 2.450 g Tadalafil Hydrochloride, 62.5 g Gelatin, 18.8 g Mannitol, 21.2 g Taurine, 0.5 g Sodium Benzoate, and 0.5 g Potassium Sorbate.

The gelatin and mannitol were shifted together until homogenous using a fork. The dry component mixture was added to 116.0 grams of water with dissolved potassium sorbate and sodium benzoate rapidly with rapid stirring. The dry mix quickly absorbed the water and became a rubbery mass. The rubbery mass was added to a zip lock bag and heated at 160° F. until free of foam and a clear yellow.

The passion fruit juice concentrate was added to a container with sucrose, cyclodextrin and glucose syrup. The components were heated until reflux until a Brix level of 87.5 was reached. The components were cooled to 200° F. and the gelatin mix was added with stirring. The temperature was maintained to remain above 180° F. A solution of 2.450 g of Tadalafil Hydrochloride in 6.100 g water was then added dropwise over a period of 30 seconds with stirring. The final Brix was 84. The solution was then added to silicone molds and the molds were placed in the refrigerator for 90 minutes.

### Example 10. Vardenafil Gummy

Ingredients include: 220.0 g Sucrose, 20.0 g beta-cyclodextrin, 185 g Glucose Syrup, 2.245 g Vardenafil Hydrochloride, 62.5 g Gelatin, 18.8 g Mannitol, 21.2 g Taurine, 0.5 g Sodium Benzoate, and 0.5 g Potassium Sorbate.

The gelatin and mannitol were shifted together until homogenous using a fork. The dry component mixture was added to 116.0 grams of water with dissolved potassium sorbate and sodium benzoate rapidly with rapid stirring. The dry mix quickly absorbed the water and became a rubbery mass. The rubbery mass was added to a zip lock bag and heated at 160° F. until free of foam and a clear yellow.

The cyclodextrin, sucrose and glucose syrup were added to a container with 50 grams of water. The components were heated until reflux until a Brix level of 87.5 was reached. The components were cooled to 200° F. and the gelatin mix was added with stirring. The temperature was maintained to remain above 180° F. A solution of 2.350 g of vardenafil hydrochloride in 6.100 g water was then added dropwise over a period of 30 seconds with stirring followed by 5 grams of grape flavor and 0.3 grams purple coloring. The final Brix was 84. The solution was then added to silicone molds and the molds were placed in the refrigerator for 90 minutes.

### Example 11. Tadalafil Gummy

Ingredients include: 220.0 g Sucrose, 20.0 g beta-cyclodextrin, 185 g Glucose Syrup, 2.550 g Tadalafil Hydrochloride, 62.5 g Gelatin, 18.8 g Mannitol, 21.2 g Taurine, 0.5 g Sodium Benzoate, and 0.5 g Potassium Sorbate.

The gelatin and mannitol were shifted together until homogenous using a fork. The dry component mixture was added to 116.0 grams of water with dissolved potassium sorbate and sodium benzoate rapidly with rapid stirring. The dry mix quickly absorbed the water and became a rubbery mass. The rubbery mass was added to a zip lock bag and heated at 160-F until free of foam and a clear yellow.

The cyclodextrin, sucrose and glucose syrup were added to a container with 50 grams of water. The components were heated until reflux until a Brix level of 87.5 was reached. The components were cooled to 200° F. and the gelatin mix was added with stirring. The temperature was maintained to remain above 180° F. A solution of 2.550 g of tadalafil hydrochloride in 6.100 g water was then added dropwise over a period of 30 seconds with stirring. 5 grams of lime flavoring and 0.6 grams of green color were added. The final Brix was 84. The solution was then added to silicone molds and the molds were placed in the refrigerator for 90 minutes.

### Example 12. Tadalafil Gummy

Ingredients include: 220.0 g Sucrose, 20.0 g beta-cyclodextrin, 185 g Glucose Syrup, 2.550 g Tadalafil Hydrochloride, 62.5 g Gelatin, 18.8 g Mannitol, 21.2 g Taurine, 0.5 g Sodium Benzoate, and 0.5 g Potassium Sorbate.

The gelatin and mannitol were shifted together until homogenous using a fork. The dry component mixture was added to 116.0 grams of water with dissolved potassium sorbate and sodium benzoate rapidly with rapid stirring. The dry mix quickly absorbed the water and became a rubbery mass. The rubbery mass was added to a zip lock bag and heated at 160-F until free of foam and a clear yellow.

The cyclodextrin, sucrose and glucose syrup were added to a container with 50 grams of water. The components were heated until reflux until a Brix level of 87.5 was reached. The components were cooled to 200° F. and the gelatin mix was added with stirring. The temperature was maintained to remain above 180° F. A solution of 2.550 g of tadalafil hydrochloride in 6.100 g water was then added dropwise over a period of 30 seconds with stirring. 5 grams of green apple was added followed by 0.6 grams of green color. The final Brix was 84. The solution was then added to silicone molds and the molds were placed in the refrigerator for 90 minutes.

### Example 13. Sildenafil Gummy

Ingredients include: 220.0 g Sucrose, 20.0 g beta-cyclodextrin, 100.0 grams powdered cocoa, 185 g Glucose Syrup, 11.0 g Sildenafil Citrate, 62.5 g Gelatin, 18.8 g Mannitol, 21.2 g Taurine, 0.5 g Sodium Benzoate, and 0.5 g Potassium Sorbate.

The gelatin and mannitol were shifted together until homogenous using a fork. The dry component mixture was added to 116.0 grams of water with dissolved potassium sorbate and sodium benzoate rapidly with rapid stirring. The dry mix quickly absorbed the water and became a rubbery mass. The rubbery mass was added to a zip lock bag and heated at 160° F. until free of foam and a clear yellow.

The cyclodextrin, cocoa powder, sucrose and glucose syrup were added to a container with 50 grams of water. The components were heated until reflux until a Brix level of 87.5 was reached. The components were cooled to 200° F. and the gelatin mix was added with stirring. The temperature was maintained to remain above 180° F. A solution of 10.6 g of sildenafil citrate in 19.5 g water was then added dropwise over a period of 30 seconds with stirring. The final Brix was 84. The solution was then added to silicone molds and the molds were placed in the refrigerator for 90 minutes.

### Example 14. Metaformin Gummy

Ingredients include: 240.0 g Sucrose, 185 g Glucose Syrup, 30.0 g Metaformin, 62.5 g Gelatin, 18.8 g Mannitol, 21.2 g Taurine, 0.5 g Sodium Benzoate, and 0.5 g Potassium Sorbate.

The gelatin, metaformin and mannitol were shifted together until homogenous using a fork. The dry component mixture was added to 130.0 grams of water with dissolved potassium sorbate and sodium benzoate rapidly with rapid stirring. The dry mix quickly absorbed the water and became a rubbery mass. The rubbery mass was added to a zip lock bag and heated at 160° F. until free of foam and a clear yellow.

The sucrose and glucose syrup were added to a container with 50 grams of water. The components were heated until reflux until a Brix level of 87.5 was reached. The components were cooled to 200° F. and the gelatin mix was added with stirring. The temperature was maintained to remain above 180° F. and 5 grams of strawberry flavor and 0.5 grams red color were added. The final Brix was 84. The solution was then added to silicone molds and the molds were placed in the refrigerator for 90 minutes.

### Example 15. Cetirizine Gummy

Ingredients include: 220.0 g Sucrose, 20.0 g beta-cyclodextrin, 200.1 g Orange Juice Concentrate 100.5 g Glucose Syrup, 1.330 g Cetirizine Dihydrochloride, 62.5 g Gelatin, 18.8 g Mannitol, 21.2 g Taurine, 0.5 g Sodium Benzoate, and 0.5 g Potassium Sorbate.

The gelatin, cetirizine dihydrochloride and mannitol were shifted together until homogenous using a fork. The dry component mixture was added to 120.0 grams of water with dissolved potassium sorbate and sodium benzoate rapidly with rapid stirring. The dry mix quickly absorbed the water and became a rubbery mass. The rubbery mass was added to a zip lock bag and heated at 160.F until free of foam and a clear yellow.

The orange juice concentrate was added to a container with sucrose and glucose syrup. The components were heated until reflux until a Brix level of 87.5 was reached. The components were cooled to 200° F. and the gelatin mix was added with stirring. The temperature was maintained to remain above 180° F. The final Brix was 84.

### Example 16. Cannabidiol Gummy

Ingredients include: 220.0 g Sucrose, 20.0 g beta-cyclodextrin, 185.0 g Glucose Syrup, 2.330 g Cannabidiol, 62.5 g Gelatin, 18.8 g Mannitol, 21.2 g Taurine, 0.5 g Sodium Benzoate, and 0.5 g Potassium Sorbate.

The gelatin and mannitol were shifted together until homogenous using a fork. The dry component mixture was added to 120.0 grams of water with dissolved potassium sorbate and sodium benzoate rapidly with rapid stirring. The dry mix quickly absorbed the water and became a rubbery mass. The rubbery mass was added to a zip lock bag and heated at 160° F. until free of foam and a clear yellow.

The sucrose, cyclodextrin, cannabidiol, and glucose syrup were added to a container with 64 grams water. The components were heated until reflux until a Brix level of 87.5 was reached. The components were cooled to 200° F. and the gelatin mix was added with stirring. The temperature was maintained to remain above 180° F. To the mixture was added 5 grams raspberry flavor as well as 0.6 grams red color. The final Brix was 84.

### Example 17. Acetylsalicylic Acid Gummy

Ingredients include: 220.0 g Sucrose, 20.0 g beta-cyclodextrin, 185 g Glucose Syrup, 14.0 g Calcium Acetylsalicylate, 62.5 g Gelatin, 18.8 g Mannitol, 21.2 g Taurine, 0.5 g Sodium Benzoate, and 0.5 g Potassium Sorbate.

The gelatin and mannitol were shifted together until homogenous using a fork. The dry component mixture was added to 116.0 grams of water with dissolved potassium sorbate and sodium benzoate rapidly with rapid stirring. The dry mix quickly absorbed the water and became a rubbery mass. The rubbery mass was added to a zip lock bag and heated at 160° F. until free of foam and a clear yellow.

The cyclodextrin, sucrose and glucose syrup were added to a container and made homogeneous. 14 grams of calcium acetylsalicylate was dissolved into 80 grams of water. The water was heated to near boiling and the sugar mixture added. The components were heated until reflux until a Brix level of 87.5 was reached. The components were cooled to 200° F. and the gelatin mix was added with stirring. The temperature was maintained to remain above 180° F. 5 grams of lime flavoring and 0.6 grams of green color were added. The final Brix was 84. The solution was then added to silicone molds and the molds were placed in the refrigerator for 90 minutes.

### Example 18. Acetaminophen Gummy

Ingredients include: 220.0 g Sucrose, 20.0 g beta-cyclodextrin, 185 g Glucose Syrup, 20.0 g Acetaminophen, 62.5 g Gelatin, 18.8 g Mannitol, 21.2 g Taurine, 0.5 g Sodium Benzoate, and 0.5 g Potassium Sorbate.

The gelatin, acetaminophen, and mannitol were shifted together until homogenous using a fork. The dry component mixture was added to 116.0 grams of water with dissolved potassium sorbate and sodium benzoate rapidly with rapid stirring. The dry mix quickly absorbed the water and became a rubbery mass. The rubbery mass was added to a zip lock bag and heated at 160° F. until free of foam and a clear yellow.

The cyclodextrin, sucrose and glucose syrup were added to a container and made homogeneous. 14 grams of calcium acetylsalicylate was dissolved into 80 grams of water. The water was heated to near boiling and the sugar mixture added. The components were heated until reflux until a Brix level of 87.5 was reached. The components were cooled to 200° F. and the gelatin mix was added with stirring. The temperature was maintained to remain above 180° F. 5 grams of lime flavoring and 0.6 grams of green color were added. The final Brix was 84. The solution was then added to silicone molds and the molds were placed in the refrigerator for 90 minutes.

### Example 19. Ibuprofen Gummy

Ingredients include: 170.0 g Sucrose, 50.0 g beta-cyclodextrin, 410 g Glucose Syrup, 12.0 g Lysine Ibuprofen, 62.5 g Gelatin, 20.0 g Mannitol, 120.0 Fructose, 0.5 g Sodium Benzoate, and 0.5 g Potassium Sorbate.

The gelatin, ibuprofen lysine, and cyclodextrin were shifted together until homogenous using a fork. The dry component mixture was added to 116.0 grams of water with dissolved potassium sorbate and sodium benzoate rapidly with rapid stirring. The dry mix quickly absorbed the water and became a rubbery mass. The rubbery mass was added to a zip lock bag and heated at 160° F. until free of foam and a clear yellow.

The fructose, mannitol, sucrose and glucose syrup were added to a container and made homogeneous. 100 grams of water was heated to near boiling and the sugar mixture added. The components were heated until reflux until a Brix level of 87.5 was reached. The components were cooled to 200° F. and the gelatin mix was added with stirring. The temperature was maintained to remain above 180° F. 2.5 grams of lime flavoring and 2.5 grams orange flavoring with 0.6 grams of orange color were added. The final Brix was 84.

The solution was then added to silicone molds and the molds were placed in the refrigerator for 90 minutes.

### Example 20. Naproxen Gummy

Ingredients include: 170.0 g Sucrose, 50.0 g beta-cyclodextrin, 410 g Glucose Syrup, 12.0 g Naproxen Sodium, 62.5 g Gelatin, 20.0 g Mannitol, 120.0 Fructose, 0.5 g Sodium Benzoate, and 0.5 g Potassium Sorbate.

The gelatin, naproxen sodium, and cyclodextrin were shifted together until homogenous using a fork. The dry component mixture was added to 116.0 grams of water with dissolved potassium sorbate and sodium benzoate rapidly with rapid stirring. The dry mix quickly absorbed the water and became a rubbery mass. The rubbery mass was added to a zip lock bag and heated at 160° F. until free of foam and a clear yellow.

The fructose, mannitol, sucrose and glucose syrup were added to a container and made homogeneous. 100 grams of water was heated to near boiling and the sugar mixture added. The components were heated until reflux until a Brix level of 87.5 was reached. The components were cooled to 200° F. and the gelatin mix was added with stirring. The temperature was maintained to remain above 180° F. 4.5 grams lemon flavoring with 0.6 grams of yellow color were added. The final Brix was 84. The solution was then added to silicone molds and the molds were placed in the refrigerator for 90 minutes.

### Example 21. Loratadine Gummy

Ingredients include: 170.0 g Sucrose, 50.0 g beta-cyclodextrin, 410 g Glucose Syrup, 1.430 g Loratadine Hydrochloride, 62.5 g Gelatin, 20.0 g Mannitol, 120.0 Fructose, 0.5 g Sodium Benzoate, and 0.5 g Potassium Sorbate.

The gelatin, loratadine hydrochloride, and cyclodextrin were shifted together until homogenous using a fork. The dry component mixture was added to 116.0 grams of water with dissolved potassium sorbate and sodium benzoate rapidly with rapid stirring. The dry mix quickly absorbed the water and became a rubbery mass. The rubbery mass was added to a zip lock bag and heated at 160° F. until free of foam and a clear yellow.

The fructose, mannitol, sucrose and glucose syrup were added to a container and made homogeneous. 100 grams of water was heated to near boiling and the sugar mixture added. The components were heated until reflux until a Brix level of 87.5 was reached. The components were cooled to 200° F. and the gelatin mix was added with stirring. The temperature was maintained to remain above 180° F. 4.5 grams lemon flavoring with 0.6 grams of yellow color were added. The final Brix was 84.

The solution was then added to silicone molds and the molds were placed in the refrigerator for 90 minutes.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the disclosure (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted y context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Embodiments of this disclosure are described herein, including the best mode known to the inventors for carrying out the disclosure. Variations of those embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

What is claimed is:

1. A method of making a gummy composition, comprising
providing a gelatin blend, comprising gelatin and a swelling agent,
providing a bonding blend, comprising a combination of at least two of a monosaccharide, a disaccharide and a tri-saccharide,
providing an active blend, comprising an active agent and a liquid solvent, wherein the active agent comprises an pharmaceutical active ingredient (API), and wherein the liquid solvent comprises a diprotic solvent, tri-protic solvent, or a combination thereof,
combining the active blend, the bonding blend and a gelatin blend to provide a molding blend wherein the bonding blend has a higher Brix number than the molding blend, and
forming the molding blend into a gummy composition.

2. The method of claim 1, wherein the swelling solvent comprises water, ethanol, glycerol or a combination thereof.

3. The method of claim 1, wherein the bonding blend consists substantially of at least three of tagatose, psicose, sorbose, trehalose, isomaltulose, mannose, or a combination thereof.

4. (canceled)

5. The method of claim 1, wherein the molding blend comprises gelatin at a concentration of about 9%.

6. The method of claim 1, wherein the active agent comprises an antibacterial agent, an antifungal agent, a pain or fever reliever, an analgesics, an anti-inflammatory agent, a steroid, a diabetic medication, an antidiarrheal agent, an antiulcer agent, a proton pump inhibitor, a laxative or cathartic agent, a diuretic agent, an antacid, an antihistamine, a decongestant, an antitussive or expectorant agent, a cough suppressant, a cold medicine, a motion sickness medication, a medication for treating alcoholism or opioid dependence, a smoke cessation agent, an anti-anxiety agent, an antidepressant, a mood stabilizer, an antipsychotic agent, a stimulant, a benzodiazepine, an anxiolytic agent, a Z-drug, a melatonergic agent, a barbiturate, an antidepressant, an antipsychotic agent, a cardiovascular agent, an anti-cancer agent, an immune suppressant, a blood thinner, an anti-diabetic agent, or a combination thereof.

7. The method of claim 6, wherein the pain or fever reliever comprises acetaminophen, aspirin, salicylic acid, ibuprofen, naproxen, or a combination thereof.

8. The method of claim 66, wherein the antihistamine comprises cetirizine, diphenhydramine, loratadine, chlorpheniramine, brompheniramine, alimemazine, cyprohetadine, doxylamine, hydroxyzine, promethazine, or a combination thereof.

9. The method of claim 6, wherein the decongestant comprises pseudoephedrine, oxymetazoline, phenylephrine or a combination thereof.

10. The method of claim 6, wherein the cough suppressant comprises dextromethorphan.

11. The method of claim 6, wherein the medication for treating alcoholism or opioid dependence comprises acamprosate, baclofen, buprenorphine, naloxone, clonidine, disulfiram, methadone, naltrexone, ondansetron, or a combination thereof.

12. The method of claim 6, wherein the smoke cessation agent comprises nicotine, bupropion, cytosine, varenicline, or a combination thereof.

13. The method of claim 6, wherein the stimulant comprises caffeine, amphetamine, dexamfetamine, lisdexamfetamine, methylphenidate, dexmethylphenidate, metamfetamine, or a combination thereof.

14. The method of claim 6, wherein the antidepressant comprises citalopram, clomipramine, dexopin, escitalopram, fluoxetine, fluvoxamine, imipramine, mirtazapine, paroxetine, sertraline, trazodone, amitriptyline, doxepin, mianserin, mirtazapine, trazodone, trimipramine, or a combination thereof.

15. The method of claim 6, wherein the antipsychotic agent comprises chlorprothizene, levomepromazine, perazine, promethazine, prothipendyl, sulpiride, thioridazine, zuclopenthixol, perphenazine, beneperidol, bromperidol, fluphenazine, fluspirilen, haloperidol, pimozide, amisulpride, aripiprazole, asenapine, chloropromazine, clozapine, flupenthixol, lloperidone, melperone, olanzapine, paliperidone, penfluridol, quetiapine, reserpine, risperidone, sertindole, thiothizene, trifluoperazine, zipraisdone, zotepine, pericyazine, carbamazepine, valproic acid, or a combination thereof.

16. The method of claim 6, wherein the cardiovascular agent comprises an aldosterone receptor antagonist, an angiotensin converting enzyme inhibitor, an angiotensin receptor blocker, a neprilysin inhibitor, an antiadrenergic agent, an antianginal agent, an antiarrhythmic agent, an anticholinergic chronotropic agent, an antihypertension agent, an ACE inhibitor, an angiotensin II inhibitor, an antiadrenergic agent, a beta blocker, a diuretic agent, a beta-adrenergic blocker, a calcium channel blocker, a catecholamine, an inotropic agent, a vasodilator, a renin inhibitor, a sclerosing agent, a vasopressin antagonist, a vasopressor, an anti-cholesterol agent, or a combination thereof.

17. A gummy composition, comprising a gelling blend, a bonding blend and an active blend, wherein the gelling blend comprise gelatin, pectin, or a combination thereof, wherein the bonding blend comprises a low GI monosaccharide and a low GI disaccharide, and wherein active blend comprises an pharmaceutical active ingredient (API).

18. The gummy composition of claim, wherein the API comprises caffeine, aspirin or its derivative, ibuprofen, naproxen, acetaminophen, diphenhydramine, cetirizine, loratadine, brompheniramine, chlorpheniramine, clemastine, fexofenadine, a PDE5 inhibitor, an anti-diabetic agent, an antibiotics, or a derivative thereof.

19. The gummy composition of claim 17, wherein the gelling blend comprises gelatin and pectin and wherein the gummy composition has a GI value of not more than 30.

20. A medication kit, comprising a gummy composition of claim 17 and an instruction for using the gummy composition.
